(19) United States
(12) Patent Application Publication
(10) Pub. No.: US 2024/0097532 A1
(43) Pub. Date: Mar. 21, 2024

(54) ELECTROMAGNETIC MOTOR FOR OPERATION IN A HIGH MAGNETIC FIELD ENVIRONMENT

(71) Applicant: University of Utah Research Foundation, Salt Lake City, UT (US)

(72) Inventors: J. Rock Hadley, Salt Lake City, UT (US); Dennis L. Parker, Salt Lake City, UT (US); Lorne W. Hofstetter, Salt Lake City, UT (US); Robb Merrill, Salt Lake City, UT (US)

(21) Appl. No.: 18/257,083

(22) PCT Filed: Dec. 16, 2021
(86) PCT No.: PCT/US21/63879

§ 371 Date: Jun. 12, 2023

Related U.S. Application Data
(60) Provisional application No. 63/126,257, filed on Dec. 16, 2020.
(60) Provisional application No. 63/215,287, filed on Jun. 25, 2021.

(51) Int. Cl. H02K 11/02; A61B 5/055; H02K 11/01; H02K 11/22; H02K 11/40; H03H 7/01

(52) U.S. Cl. CPC H02K 11/02; A61B 5/055; H02K 11/0141; H02K 11/22; H02K 11/40; H03H 7/0115; A61B 34/30

(57) ABSTRACT

A mechanically commutated motor (200) configured for use with an external magnetic field (240) is disclosed. The motor (200) includes an axle (208) formed of anon-magnetic material. A rotor (206) is coupled to the axle (208), the rotor (206) including three or more actuator units (205) spaced about the axle (208). Each actuator unit (205) comprises a non-magnetic material, a coil winding (214) along each of the three or more actuator units (205), and a commutator (216) coupled to the axle (208) and electrically associated with the coil windings (214). The motor (200) further includes two or more resilient contacts (210) oriented to direct a current through the commutator (216) to one of the coil windings (214) to induce a current in the coil winding (214) to form an electromagnet that rotates the rotor (206) relative to the external magnetic field (240) from a magnet located external to the mechanically commutated motor (200).

### RELATED APPLICATIONS

This application claims the benefit of U.S. Provisional Patent Application No. 63/126,257, filed Dec. 16, 2020 and U.S. Provisional Patent Application No. 63/215,287, filed Jun. 25, 2021 which are each hereby incorporated herein by reference in their entirety.

### GOVERNMENT INTEREST

This invention was made with government support under Grant CA228363 awarded by the National Institutes of Health. The government has certain rights in this invention.

### BACKGROUND

Magnetic resonance imaging (MRI) can perform highly detailed and accurate soft-tissue imaging. The use of MRI with the precision of robotic-assisted surgeries has the potential to revolutionize image-guided neurosurgeries, tissue biopsies, prostate cancer brachytherapy treatments, other soft-tissue image-guided surgeries, and diagnostic applications requiring the use of mechanical actuation. However, strong magnetic fields generated by the MRI limit use of conventional robotic servos and stepper motor actuators near the imaging region. Conventional electromagnetic motors experience significant forces in the MRI from their ferromagnetic components making them potential projectile hazards. The ferromagnetic actuator components in a conventional motor can also degrade image quality by disrupting the magnetic field homogeneity. Electrical currents in lead wires and transmitted across electrical contacts can introduce radio frequency noise into the shielded scanner room and this radiofrequency noise source can degrade image quality.

MRI can volumetrically image the human body in a non-invasive manner without the use of ionizing radiation. Its ability to visualize anatomical structure and pathology of soft tissues in exquisite detail, as well as provide functional information, have made its use instrumental for the preoperative surgical planning of neurosurgeries, orthopedic procedures, tissue biopsies, and cancer therapies. However, images acquired preoperatively can quickly become outdated due to patient motion and procedure induced changes in the tissue environment. The introduction of needles, resection of tissues, or performing a craniotomy to gain surgical access to the brain can result in shifting and deformation of soft tissues in the area of interest. This tissue shift is particularly problematic for procedures where targeting accuracy is paramount in achieving a favorable outcome or when intraprocedural discrimination between diseased and healthy tissue relies on advanced imaging techniques such as MRI.

The development of intraoperative MRI emerged to address limitations associated with using static preoperative imaging for surgical guidance. In 1994, an open 0.5 Tesla (T) MRI design was introduced that allowed direct surgical access to the patient during imaging. The benefits of this surgical approach quickly became apparent in the resection of glioma brain tumors where maximally resecting the tumor while preserving eloquent brain regions has been shown to improve survival. More recently, the improved image resolution and widespread availability of closed-bore and high-field scanners (1.5-T and 3-T) has driven their use for intraoperative MRI. However, the closed-bore nature of these systems (60-70 cm bore diameter) limits surgical access to the patient during imaging. Freehand approaches are possible but are ergonomically difficult and can involve a physician reaching up to 1 meter into scanner bore for access. As a work-around, patient transport to the imaging region of the MRI or operating rooms equipped with a mobile MRI system are used intraoperatively to confirm critical steps during a variety of procedures. However, this paradigm of move-to-image is reactionary and does not easily enable concurrent intraoperative imaging.

To compensate for limited patient access in closed-bore MRI scanners, medical robotic systems have been developed that can operate safely in the scanner bore. The aim of such systems is to combine the precision of robotic-assisted procedures with the clinical benefits of high resolution intraoperative MRI. However, design of these medical systems is complicated by the strong magnetic field generated by the superconducting magnet of the MRI system. Useful robotic systems typically involve the use of multiple motors and actuators. Traditional electromagnetic servomotor actuators that have been refined and vetted over decades of use in industrial automation and commercial medical robots are inherently incompatible with MRI. Ferromagnetic and magnetic material used by conventional electromagnetic actuators can become dangerous projectiles if brought near the magnetic field of the MRI scanner. Hence, to date, medical robots that can operate in the MRI have relied on non-magnetic pneumatic and piezoelectric actuator technologies. However, the limited accuracy of pneumatically controlled actuators that utilize long transmission lines and the potential for significant oscillation and overshoot make their use unsuitable where high precision is paramount. The electromagnetic noise generated by the operation of commercially available piezoelectric actuators can interfere with the sensitive receiver hardware of the MRI and actuator operating during imaging has been shown to reduce image signal to noise (SNR) by 26-80%. While the use of specially designed controllers have been used to keep this SNR degradation to below 15%, achieving dynamic and smooth proportional actuation via closed-loop control of piezoelectric actuators is not trivial. The inability to use the electromagnetic actuation principles that are mainstays of industrial automation has limited the development, functionality, and adoption of medical systems that combine the benefits of robotic precision with the capabilities enabled by high-resolution intraoperative MRI.

### SUMMARY

Electromagnetic motors can enable useful functions to be performed near high magnetic field environments, such as robotic assisted surgery within medical resonance imaging (MRI) systems, exciting strain waves in tissues as part of diagnostic elastography imaging protocols, positioning and orienting transducers in MR guided focused ultrasound therapies, cannula placement for deep brain neurosurgeries or tissue biopsy, or for mechanical functions near other types of superconducting magnetic systems.

Various configurations of electromagnetic motors and servo motors are disclosed herein that are substantially comprised of non-magnetic materials. Direct current motors are disclosed. Servo motors comprised of the direct current motor are also disclosed. Mechanisms for minimizing electromagnetic interference between the electromagnetic motors and the high magnetic environments are also disclosed. The use of an electromagnetic motor as a generator to measure the mechanical output of a patient is also disclosed. Applications where an electromagnetic servomotor is used to provide a mechanical excitation source for tissue stiffness quantification imaging protocols are also disclosed. Additional mechanisms for providing motor control and feedback are also disclosed.

In one example, a mechanically commutated motor, composed entirely of non-magnetic materials, can be configured for use with an external magnetic field. The mechanically commutated motor can include an axle comprising a non-magnetic material. A rotor can be coupled to the axle and can include actuator units, coil windings, a commutator, one or more bearings, a motor case, and two or more resilient contacts and all can comprise non-magnetic materials. Three or more actuator units can be spaced about the axle and each actuator unit can comprise a non-magnetic material. Coil windings can be oriented along each of the three or more actuator units. Typically, these coil windings can be electrically independent from one another, such that current flowing through one coil does not also electrically flow through another (e.g. even though there will be some inductive effects in adjacent coils). A commutator can be coupled to the axle and electrically associated with the coil winding. The commutator can consist of brushes that are connected to the stationary electric leads of the motor. The brushes can mechanically contact one or more conducting segments that rotate with the rotor. The conducting segments can be separated by an air gap or insulator. The geometric orientation of the conducting segments with respect to the brushes and rotor armature windings is chosen such that the electrical currents supplied to the motor leads are distributed to the rotor armature to achieve the desired mechanical output. A motor case can also surround the rotor, and can comprise a non-magnetic material. The non-magnetic material surrounding the motor case can electrically conducting to minimize the production of radio frequency noise external to the motor housing. The radio frequency energy produced external to the motor housing can also be reduced by choosing the axle or the portion of the axle of the motor, which extends beyond the motor housing to be made from a low-electrical conductivity material such as fiberglass, carbon fiber, or titanium. Two or more resilient contacts (e.g. brushes) oriented to direct a current through the commutator to one of the coil windings can induce a current in the coil winding to form an electromagnet in the coil winding and a corresponding magnetic field that is configured to rotate the rotor relative to an external magnetic field from a magnet located external to the motor case. A non-magnetic encoder or one or more magnetic field sensors can provide information about the rotor position to a motor controller, which can use the position information to operate the mechanically commutated motor as a servo motor.

There has thus been outlined, rather broadly, the more important features of the invention so that the detailed description thereof that follows may be better understood, and so that the present contribution to the art may be better appreciated. Other features of the present invention will become clearer from the following detailed description of the invention, taken with the accompanying drawings and claims, or may be learned by the practice of the invention.

### BRIEF DESCRIPTION OF THE DRAWINGS

FIG. 1 is an end view of a 3-pole direct current (DC) motor utilizing two permanent magnets in accordance with prior art;

FIG. 2a is a schematic diagram of a high magnetic field compatible mechanically commutated electromagnetic motor that is driven by a current and is configured for operation within an externally produced high magnetic field in accordance with an example;

FIG. 2b is an image of an exploded view of a high magnetic field compatible mechanically commutated electromagnetic motor with a non-magnetic enclosure in accordance with an example;

FIG. 2c is the high magnetic field compatible mechanically commutated electromagnetic motor of FIG. 2b located in the non-magnetic enclosure in accordance with an example;

FIG. 3 is an image of components for a high magnetic field compatible mechanically commutated electromagnetic motor in accordance with an example;

FIG. 4 is an image of a high magnetic field compatible mechanically commutated electromagnetic motor in accordance with an example;

FIG. 5a is an image of the high magnetic field compatible mechanically commutated electromagnetic motor operating in a magnetic resonance imaging (MRI) system in accordance with an example;

FIG. 5b is a zoomed image of the high magnetic field compatible mechanically commutated electromagnetic motor of FIG. 5a showing the spinning rotor and axle and the lead wires in accordance with an example;

FIG. 6 is an integrated motor system with a motor controller wired to the high magnetic field compatible mechanically commutated electromagnetic motor and a shielding system in accordance with an example;

FIG. 7 is an H-bridge controller for the motor controller with a passive filtering scheme in accordance with an example;

FIG. 8a is a high level diagram of a feedback controlled position controller with position encoder for an external high magnetic field compatible mechanically commutated electromagnetic servo motor in accordance with an example;

FIG. 8b is a is a diagram of an MRI system with shielded motors and controllers in accordance with an example;

FIG. 9a is an illustration of a portion of the components in an external high magnetic field compatible mechanically commutated servo motor in accordance with an example;

FIG. 9b is an illustration of a polycarbonate housing for the motor of FIG. 9a in accordance with an example;

FIG. 9c is an illustration of a housing end of the polycarbonate housing of FIG. 9b in accordance with an example;

FIG. 9d is an illustration of an end ring with brushes configured to be attached to the commutator of the motor of FIG. 9a in accordance with an example;

FIG. 9e is an illustration of an end ring configured to be attached to the side opposite the commutator of the motor of FIG. 9a in accordance with an example;

FIG. 9f is an illustration of encoder sensors to be coupled in the polycarbonate housing of FIG. 9b in accordance with an example;

FIG. 9g is an illustration of an encoder wheel to be used with the encoder sensors of FIG. 9f in accordance with an example;

FIG. 10a is an block diagram of a position controller with encoder for an external high magnetic field compatible mechanically commutated electromagnetic servo motor in accordance with an example;

FIG. 10b is an illustration of an assembled view of the components of the external high magnetic field compatible mechanically commutated electromagnetic servo motor illustrated in FIGS. 9a-g in accordance with an example;

FIG. 10c is an illustration of a side view of the encoder wheel and encoder sensors of FIG. 10b in accordance with an example;

FIG. 10d is an illustration of a servomotor controller of the external high magnetic field compatible mechanically commutated electromagnetic servo motor in accordance with an example;

FIG. 10e is an illustration of an external high magnetic field compatible mechanically commutated servo motor system including a motor controller and shielded cable in accordance with an example;

FIG. 11 is a schematic of a servomotor optical encoder circuit using an Arduino controller in accordance with an example;

FIG. 12 is a chart showing a signal to noise ratio (SNR) for a shielded external high magnetic field compatible mechanically commutated servo motor with a non-conducting axle and an unshielded motor in accordance with an example;

FIG. 13 is a chart showing a measured signal to noise ratio (SNR) of images acquired using an MRI relative to an SNR of a control image (SNRc) at different distances in accordance with an example;

FIG. 14 is a diagram of an external high magnetic field compatible mechanically commutated electromagnetic servo motor coupled to an unbalanced weight to form a mechanical excitation source device to be used for magnetic resonance elastography (MRE) in accordance with an example;

FIG. 15a is a diagram of an external high magnetic field compatible mechanically commutated electromagnetic servo motor used in a single degree-of-freedom biopsy introducer robot prior to introducer insertion in accordance with an example;

FIG. 15b is a diagram of the biopsy introducer robot of FIG. 15a with the biopsy introducer fully extended in accordance with an example;

FIG. 16a is an illustration of the biopsy introducer robot of FIG. 15a prior to introducer insertion into a tissue sample in accordance with an example;

FIG. 16b is an illustration of the tissue sample of FIG. 16a prior to insertion of the biopsy introducer in accordance with an example;

FIG. 16c is an illustration of the biopsy introducer robot of FIG. 16a with the cutting stylet and introducer fully inserted into the tissue sample in accordance with an example;

FIG. 16d is an illustration the tissue sample of FIG. 16a with the cutting stylet and introducer fully inserted into the tissue sample in accordance with an example;

FIG. 16e is an illustration of the biopsy introducer robot of FIG. 16a with the cutting stylet withdrawn while maintaining the position of the introducer fully inserted into the tissue sample in accordance with an example;

FIG. 16f is an illustration of the tissue sample of FIG. 16a with the introducer sheath fully inserted and the cutting stylet withdrawn in accordance with an example;

FIG. 16g is an illustration of the tissue sample prior to insertion of the introducer by the biopsy introducer robot of FIG. 16a; and

FIG. 16h is an illustration of the tissue sample with the insertion of the introducer by the biopsy introducer robot of FIG. 16a and the cutting stylet withdrawn in accordance with an example.

These drawings are provided to illustrate various aspects of the invention and are not intended to be limiting of the scope in terms of dimensions, materials, configurations, arrangements or proportions unless otherwise limited by the claims.

### DETAILED DESCRIPTION

While these exemplary embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration only and not as a limitation to describe the features and characteristics of the present invention, to set forth the best mode of operation of the invention, and to sufficiently enable one skilled in the art to practice the invention. Accordingly, the scope of the present invention is to be defined solely by the appended claims.

### Definitions

In describing and claiming the present invention, the following terminology will be used.

The singular forms “a,” “an,” and “the” include plural referents unless the context clearly dictates otherwise. Thus, for example, reference to “a terminal” includes reference to one or more of such materials and reference to “rotating” refers to one or more such steps.

As used herein, the term “about” is used to provide flexibility and imprecision associated with a given term, metric or value. The degree of flexibility for a particular variable can be readily determined by one skilled in the art. However, unless otherwise enunciated, the term “about” generally connotes flexibility of less than 2%, and most often less than 1%, and in some cases less than 0.01%.

As used herein with respect to an identified property or circumstance, “substantially” refers to a degree of deviation that is sufficiently small so as to not measurably detract from the identified property or circumstance. The exact degree of deviation allowable may in some cases depend on the specific context.

As used herein, “adjacent” refers to the proximity of two structures or elements. Particularly, elements that are identified as being “adjacent” may be either abutting or connected. Such elements may also be near or close to each other without necessarily contacting each other. The exact degree of proximity may in some cases depend on the specific context.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

As used herein, the term “at least one of” is intended to be synonymous with “one or more of” For example, “at least one of A, B and C” explicitly includes only A, only B, only C, and combinations of each.

Concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given herein.

### MRI Compatible Actuators

Implementing motorized systems near devices with strong magnetic fields, such as in an MRI environment or near superconducting magnetic systems, introduces a number of fundamental engineering challenges. MR machines and other systems that use superconducting magnets typically operate within a magnetic field that can be thousands of times stronger than the earth's magnetic field. Electric motors have many magnetic components, typically including a ferromagnetic rotor, a stator with permanent magnets, a steel housing, and so forth. At best, the strong magnetic field of the MR machine can provide high levels of force on the magnetic components of an electric motor. At worst, the magnetic and ferromagnetic components in an electric motor can become potentially lethal projectiles in the strong magnetic field.

Electromagnetic machines that are safe to use next to the patient in an MRI scanner can be used for useful interventional or diagnostic applications. An electromagnetic motor can be used as a generator to measure the physical mechanical output from a subject while they perform tasks in the MRI scanner. An electromagnetic actuator can also be used to generate a mechanical disturbance in tissue to enable MRI imaging to be performed to measure tissue elastic properties. The electric machines and motors described herein may be useful for these applications as well as surgical applications. While an MRI system is used herein as examples for operating an electromagnetic motor near an external high magnetic field, it is not intended to be limiting. The electromagnetic motor can be configured to operate near any external magnet with a high magnetic field, such as a superconducting magnet, electromagnet or permanent magnet, that has sufficient magnetic force to drive the motor. The terms “high magnetic field” or “strong magnetic field” are intended to include magnetic fields of greater than approximately 0.1 Tesla (T), in some cases from 2-10 T, and in some cases greater than about 20 T.

Examples are provided for electromagnetic motor and servo motor designs that can both operate near a high magnetic field, such as inside an MRI machine near a patient or within or near a fusion reactor. The electromagnetic motor can utilize the strong magnetic field produced by the superconducting magnet. One aspect of these motor configurations includes using magnetic fields produced by superconducting magnets within MRI systems to generate forces on the rotor windings. In one example, the rotor can be configured to be free to rotate any number of revolutions with no ratcheting mechanism or conversion or oscillatory motion to rotational motion involved. The magnetic fields produced by the MRI can be used to generate forces in conducting wires in the motor rotor.

In another example, the electromagnetic motor and servo motor designs disclosed herein can be configured to operate in other environments that use superconducting magnets, such as particle accelerators and fusion power systems. Superconducting magnets with magnetic field strengths greater than 20 T are being developed for fusion power systems. Robotic systems can be used in a fusion power system or particle collider system to reduce the need for human interaction with each type of system while it is operating.

In one example, the magnetic fields produced by the system can be configured to perform as the stator field of the motor. Unlike conventional electromagnetic motors, the example design does not use ferromagnetic or magnetic components in the motor. Materials with magnetic or ferromagnetic components that are placed near an external high magnetic field, such as the magnetic field within an MRI, can become a projectile hazard. In addition the magnetic field produced by a permanent magnet in a traditional motor design can be counter-acted by the magnetic fields produced by the MRI, which may also limit the functionality of traditional motors in the MRI even if properly fastened to prevent the possibility of becoming a projectile.

Several different motor design variations are disclosed herein. In one example, a mechanically commutated motor and servo motor design driven by a direct current (DC) including variable current output from a motor controller (e.g. pulse width modulation) can use mechanical commutation (i.e. brushes) to supply currents to conducting loops in the rotor. The rotor and motor design is free from ferromagnetic or magnetic components. For a mechanically commutated servo motor design, encoding of a rotor position can be achieved using MRI-compatible methods. Information about the rotor positions can be returned to the motor controller and provide feedback information used for servo control algorithms. To allow a mechanically commutated motor and servo motor design to be operated simultaneous to an MR imaging machine's operation, a variety of novel radio frequency (RF) noise reduction strategies have been developed and integrated in the mechanically commutated motor/servo system to reduce MR image quality degradation that the mechanically commutated motor, motor controller generated currents, or servo feedback may cause when the motor is in operation. These aspects of the invention are described in detail below.

In addition to outlining these high magnetic field compatible electromagnetic motor technologies, servo motor designs by combining motor configurations (1) with an encoder and control hardware are described herein. In addition, certain types of high magnetic field environments, such as an MRI system, can be extremely sensitive to radio frequency noise. The simultaneous operation of electrical systems during imaging can degrade imaging quality produced by the MRI system. Approaches are disclosed for shielding and/or controlling the high magnetic field compatible motors in such a way to allow for simultaneous (or interleaved) imaging and motor actuation while maintain suitable image quality. An interleaved operation can comprise alternating repetition time (TR) of MR scanning between motor actuation and MR imaging. For modern imaging protocols, the TR can be as short as 5 milliseconds (ms). So rapid switching between motor operation and imaging can allow for concurrent motor actuation and imaging generation.

Standard DC Motor with Mechanical Commutation

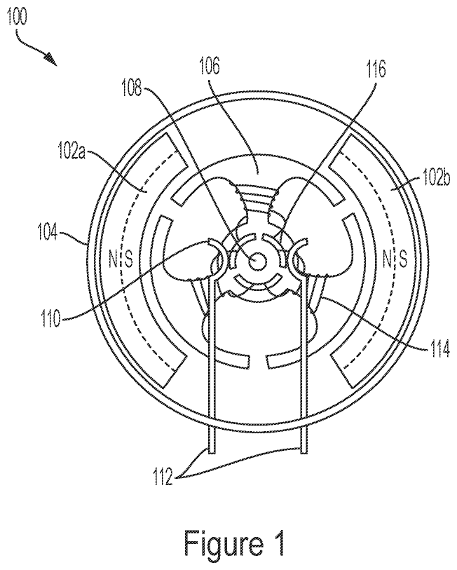

A brief introduction to the basics of standard direct current (DC) motor design is presented to elucidate differences between a standard DC motor design and DC motors configured to operate in high magnetic fields that are produced externally to the motor. The schematic of a standard and widely used permanent magnet DC motor 100 is illustrated in FIG. 1, which shows an end view of a 3-pole DC motor utilizing two permanent magnets 102a, 102b to generate the stator field. Several aspects of this motor make it incompatible with a typical MRI environment. Permanent magnets 102a, 102b and the ferromagnetic motor housing 104 and ferromagnetic rotor 106 (used to focus the magnetic flux) are critical components of the design of the DC motor illustrated in FIG. 1. However, these components are, by design, highly magnetic. The ferromagnetic motor housing 104 is often made of magnetic steel as well. The purpose of the ferromagnetic rotor 106, such as a steel rotor, is to help focus magnetic flux and improve motor efficiency. The rotor 106 can also be made from ferromagnetic laminations which focus the magnetic flux and thereby enhance the torque generation between rotor windings 114 and the permanent magnets 102a, 102b.

The brushes 110, terminals 112, and commutator 116 can also be formed of a ferromagnetic material. These magnetic components can become a projectile hazard if they are brought near a strong magnetic field, such as the field in an MRI environment. In addition, the strong magnetic fields of the MRI can also prevent proper functioning of these motors. The magnetic fields produced by the MRI system can overwhelm or distort the direction and magnitude of the magnetic field produced by the permanent magnets 102a, 102b in the motor 100, which can significantly affect the operation of the motor in a strong magnetic field environment.

### (1) High Magnetic Field Compatible Mechanically Commutated Motor

In accordance with one example, embodiments of a high magnetic field compatible motor and a servo motor have been developed by making several modifications to a conventional DC motor. Differentiating factors include: (i) the magnetic rotor is replaced with a non-magnetic support structure for the rotor windings. (ii) The permanent magnets are removed from the motor design and the ambient external high magnetic field becomes the stator field of the motor. A geometry of the commutation rings and rotor windings are chosen such that the magnetic field generated by a high magnetic field system external to the motor, such as an MRI system, can be used in place of permanent magnets 102a, 102b illustrated in FIG. 1. (iii) The stator housing and/or all motor support structures are either removed or replaced with non-magnetic materials.

One element for a material to be considered to be non-magnetic is that it is not ferromagnetic. Non-limiting examples of metals that are ferromagnetic include iron, cobalt, nickel, chromium, and manganese. These metals are not considered to be non-magnetic. Metals such as copper, zinc, and aluminum are either diamagnetic or so weakly paramagnetic that they are essentially considered non-magnetic. There are some stainless steels such as 316LVM that are weakly paramagnetic and can be considered to be essentially non-magnetic. To be specific, a material is considered to be non-magnetic in a high magnetic field environment, such as an MRI environment, if its magnetic susceptibility χ meets the following conditions |χ_{w}−χ|<10⁻¹ where χ_{w} is the magnetic susceptibility of water. (iv) A systems level design that reduces the production of RF noise near the Larmor frequency. The reduction of electromagnetic interference can enable the mechanically commutated motor to be operated simultaneously with MR imaging. The electromagnetic interference (EMI) reduction can be accomplished using design aspects to reduce RF noise generated by the mechanical commutation, lead wires, and motor controllers.

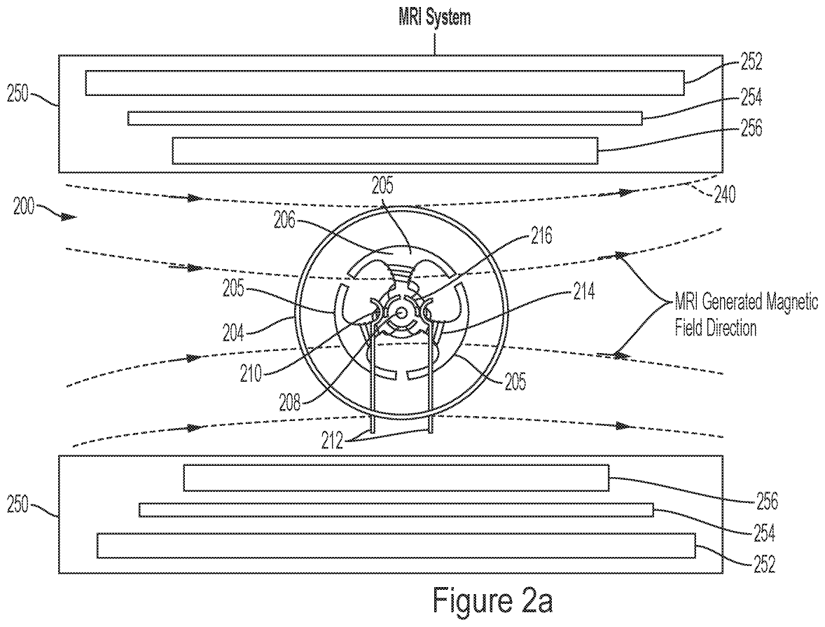

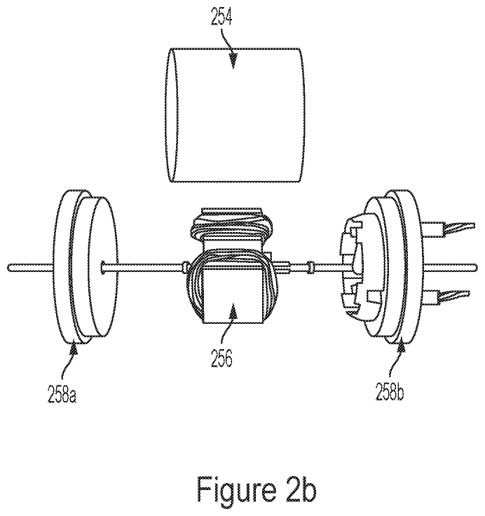

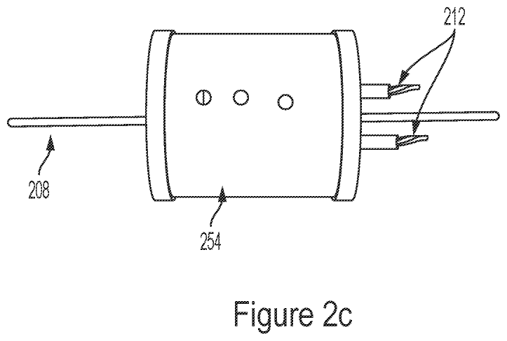

FIG. 2a illustrates a schematic diagram of an external high magnetic field compatible mechanically commutated electromagnetic motor 200 configured for operation within an externally produced high magnetic field 240 in accordance with an example. The external high magnetic field compatible mechanically commutated electromagnetic motor concept (motor concept) can operate within an ambient high magnetic field, such as near the patient area of MRI systems. The motor concept does not necessitate the use of permanent magnets or a ferromagnetic rotor. The motor concept is designed to utilize an external high magnetic field, such as the magnetic field of a superconducting magnet of an MRI scanner which obviates the need for permanent magnets. Since the magnetic field is homogeneous, strong (1.5-3T for standard clinical systems), and extends well beyond the patient area, the use of a ferromagnetic rotor to focus magnetic flux is unnecessary to maintain a strong magnetic flux density in the air gap. Removing the ferromagnetic rotor has additional benefits in that it eliminates unwanted motor cogging torque that is associated with the reluctance of the ferromagnetic materials. Reduction in motor cogging can be particularly important for robotic applications that use high precision.

In the example of FIG. 2a, the externally produced high magnetic field 240 is provided by an MRI system 250 that includes a high field magnet 252 located external to the mechanically commutated motor 200. The MRI system 250 can also include a gradient coil 254, and a body RF coil 256. The gradient coil 254 is comprised of loops of wire or thin conductive sheets on a cylindrical shell that lies just inside the bore of an MRI system 250. When an electrical current passes through these coils, the result is a secondary magnetic field. This gradient field distorts the main magnetic field 240 in a slight but predictable pattern. The pattern can be modulated to cause a resonance frequency of protons to vary as a function of position, which can be used for spatial encoding in one or more dimensions. The body coil acts as an antenna to receive the RF signal coming out of a body and transmits that data to a computer which can use the data to generate images.

In the example of FIG. 2a, the external high magnetic field compatible mechanically commutated motor 200 includes a rotor 206 that is coupled to an axle 208. The rotor 206 can be comprised of three or more actuator units 205 that are spaced about the axle 208. A coil winding 214 is positioned along each of the three or more actuator units 205. The coil windings 214 can comprise a conductive material such as, but not limited to, one or more of copper, aluminum, silver, or gold or a mixture thereof. The rotor 206 and axle 208 are comprised of a non-magnetic material selected to support the coil windings 214. No permanent magnets are used. The main magnetic field 240 of the MRI system 250 is used to generate the stator magnetic field. This stator field interacts with electric current in the rotor coil windings 214 to generate a force on one or more coil windings 214 to rotate the rotor 206 and corresponding motor axle 208. Commutation can be achieved mechanically in this example using a commutator 216 that is coupled to the axle 208 and electrically associated with one or more of the coil windings 214.

Mechanical commutation is achieved with the commutator 216 and brushes 210 that are connected to the stationary terminals (electric leads) of the motor 200. The brushes 210 can mechanically contact one or more conducting segments that rotate with the rotor 206. The conducting segments are separated by an air gap or insulator. The geometric orientation of the conducting segments with respect to the brushes 210 and rotor armature windings 214 is chosen such that the electrical currents supplied to the motor leads connected to the terminals 212 are distributed to the rotor armature to achieve the desired mechanical forces on the rotor 206.

A voltage is applied across the terminals 212 to power the motor 200. In one example, the terminals 212 end in two or more resilient contacts (e.g. brushes 210) that are oriented to direct a current through the commutator 216 to one of the coil windings 214 to induce a current in the coil winding 214 to form an electromagnet that is configured to rotate the rotor 206 relative to the external high magnetic field 240 from the high field magnet 252 located external to the mechanically commutated motor 200.

Both clockwise and counterclockwise rotation of the rotor 206 can be achieved by switching the polarity of the voltage applied to the terminals 212. The motor speed or acceleration can be adjusted as well by varying the magnitude of the terminal voltages. A motor controller, such as an H-bridge motor controller, can be used for precise control of the motor 200. Other types of motor controllers can be used as well, as can be appreciated.

A non-magnetic motor case (housing) 204 is illustrated in the diagram of FIG. 2a as well. The motor 200 and terminals 212 can be properly shielded to minimize RF noise generated by the motor 200 which can degrade MR image quality if imaging is performed during motor operation. To add additional noise-reduction capabilities to this design, the non-magnetic motor case 204 can be electrically conducting.

In one example, the motor 200 can be fully enclosed in a non-magnetic conductive faraday cage. However, any mechanical shaft that is made from electrically conducting materials can act as an antenna and radiate electromagnetic noise outside of the faraday cage. To enable simultaneous servomotor and MRI operation without degrading image quality, a non-conducting material can be used for the motor axle 208. In general any material having sufficiently low conductivity (e.g. polymer, carbon fiber, titanium, FR4 Garolite, fiberglass, and others) can work. Generally, a Faraday cage can be used to shield noise produced from an external high magnetic field compatible mechanically commutated electromagnetic motor. The motor axle (or output of a gearbox) that penetrates the faraday cage can be constructed from a material with sufficiently low conductivity that the axle or gear box will not behave as an antenna and will not radiate electromagnetic energy from the operation of the motor 200 outside of the faraday cage.

The radio frequency energy produced external to the motor housing 204 can be reduced by choosing the axle 208 or the portion of the axle 208 of the motor 200, that extends beyond the motor housing 204 to be made from a low-electrical conductivity material such as a polymer, fiberglass, carbon fiber, or titanium. A small capacitor between each motor terminal and the motor casing can be connected to help reduce and eliminate broadband noise generated by the brushes of the mechanical commutators. Alternatively, the capacitor between the motor terminals or motor casing may not be needed in selected embodiments.

FIG. 2b shows an exploded view of an external high magnetic field compatible mechanically commutated motor 200 with a non-magnetic enclosure. An example enclosure body 254 is illustrated that can surround the rotor with windings 256 and be coupled to two enclosure ends 258a, 258b. One of the enclosure ends 258b can include the brushes 210 and electrical terminals 212. A motor controller can be coupled to the electrical terminals 212 illustrated in FIG. 2c, which illustrates the mechanically commutated motor with the non-magnetic enclosure 254 with the axle 208 extending beyond the enclosure.

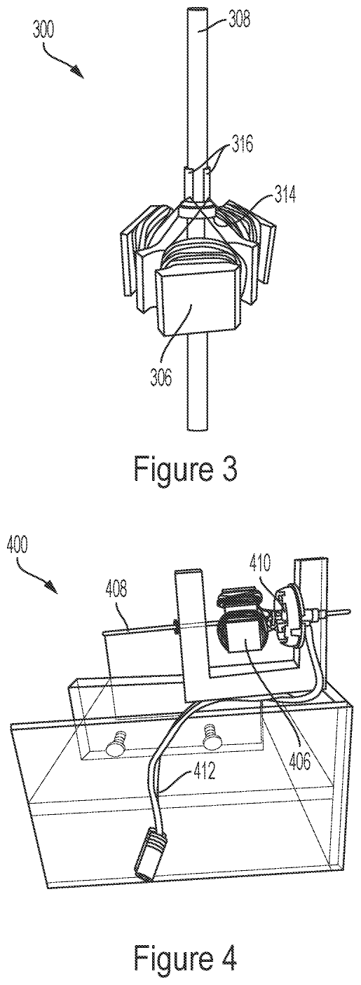

FIG. 3 is an example illustration of components of an external high magnetic field compatible mechanically commutated electromagnetic motor 300. The components illustrated include an axle 308, commutator 316, windings 314, and non-magnetic rotor 306. FIG. 4 is an example illustration of an external high magnetic field compatible mechanically commutated electromagnetic motor 400, including the non-magnetic rotor 406, the axle 408, and also illustrating the terminals 412 and brushes 410 of the commutator 316 illustrated in FIG. 3. In this example, the terminals 412 have been extended using conductors attached to the electrical terminals 412 of FIG. 2c. The conductors used to extend the terminals 412 can be formed from a non-magnetic conductive material, as previously discussed. In addition, the terminals 412 can be shielded to minimize radio frequency emissions from the terminals 412. This will be discussed more fully in the proceeding paragraphs.

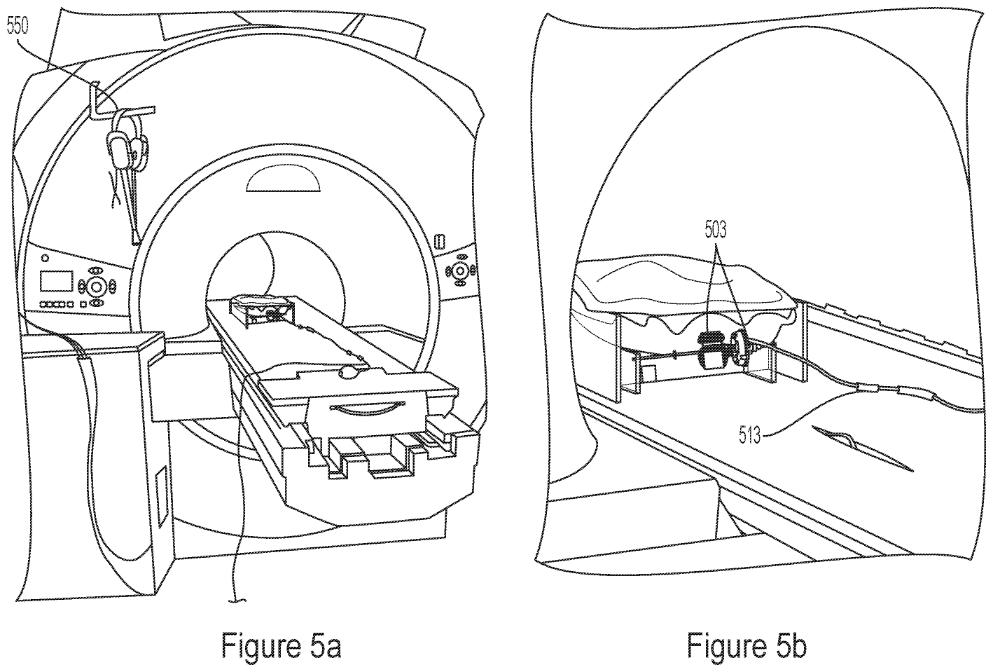

FIG. 5a is an image of the external high magnetic field compatible mechanically commutated electromagnetic motor 400 of FIG. 4 that is operating in a magnetic resonance imaging (MRI) system 550. FIG. 5b is a zoomed image of the external high magnetic field compatible mechanically commutated electromagnetic motor 400 of FIG. 4, showing the spinning rotor and axle 503 and lead wires 513 coupled to the terminals 412 (FIG. 4) in accordance with an example.

### Radio-Frequency Noise Reduction

In an MRI scanner 550, the Larmor frequency is a critical frequency at which the protons precess. Any RF noise having a frequency near the Larmor frequency can corrupt the signal that the MRI system uses for imaging. Noise reduction strategies further enable simultaneous MR imaging and motor operation in applications where the RF noise sources near the Larmor frequency are further eliminated.

As previously discussed, certain types of high magnetic field environments, such as the MRI environment, can be extremely sensitive to radio-frequency (RF) noise. Using brushed mechanical commutation, as described in the mechanical commutation motor design described previously, can lead to significant RF noise that can occur as each brush mechanically engages and disengages with a commutator segment connected to a motor winding. The RF noise can degrade the MR imaging quality when the high magnetic field compatible mechanically commutated electromagnetic motor 400 (FIG. 4) is in operation. A motor controller configuration, such as an H-bridge controller configuration, can be used to control the direction, acceleration, and speed of the motor. An H-bridge configuration is an electronic circuit configuration that switches the polarity of a voltage applied to a load. A change in the polarity can be used to drive the motor forwards or backwards. The H-bridge configuration can also be used to vary the time-average current to a load. However, pulse width modulation (PWM) switching schemes used by these H-bridge circuits create RF energy over a wide range of frequencies that can overlap with the Larmor frequency used by MRI systems. This noise from the PWM signal can also affect imaging. Lead wires connecting the motor to the motor controller can act as antennas that radiate or absorb RF energy produced by the MRI or data communicated within the wires.

An integrated design for a high external magnetic field compatible mechanically commutated electromagnetic motor and motor controller is disclosed that significantly attenuates unwanted RF radiation in the imaging region of the MRI scanner. Both passive circuit elements and shielded components can be used to reduce the production and radiation of unwanted RF noise. This combination of passive filtering and shielded components can sufficiently eliminate the RF noise near the Larmor frequency of the MRI scanner so that simultaneous imaging and motor actuation can be achieved with minimal degradation to the quality of the MR images.

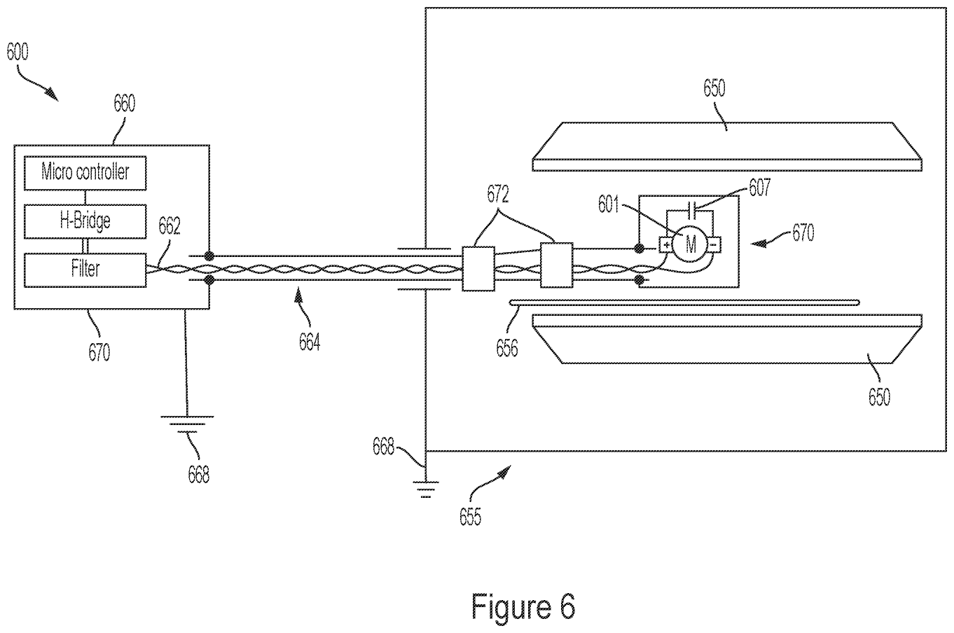

A high level view of an external high magnetic field compatible mechanically commutated electromagnetic motor 601, motor controller 660, and RF noise reduction aspects are presented in FIG. 6 in an example in which the integrated external high magnetic field compatible mechanically commutated motor system 600 is used in an MRI system 650 at high magnetic field that is external to the motor system 600. The integrated mechanically commutated motor system 600 comprises a motor controller 660, which can be located inside or outside of the MR scanner room 655 and does not need to be in the bore of the MR scanner 650 or in the shielded MR scanner room 655. The external high magnetic field compatible mechanically commutated electromagnetic motor 601, which was described in preceding paragraphs, is illustrated as located on a patient table 656 located in the bore of the MR scanner 650. The motor 601 is configured to operate in an external high magnetic field environment, such as the bore or near the bore of the MR scanner 650. Both the external high magnetic field compatible mechanically commutated electromagnetic motor 601 and the motor controller 660 can be enclosed by a conducting shielded enclosure 670 or other form of a Faraday cage. The output of the motor controller 660 can be carried on two conducting wires 662 that travel down a shielded path, such as a twisted shielded cable 664 or coaxial cable to the high external magnetic field—compatible motor 601. The shield 670 of the wires 662, motor controller 660, and housing of the motor 601, may all be electrically connected and grounded 668. This shielding 670 helps prevent RF noise generated by the motor controller 660, motor 601, and signals carried on the lead wires 662 from being radiated into the MRI environment within the room 655.

In one embodiment, a capacitor 607 connecting the two terminals of the motor 601 can be used to reduce RF noise generated by the brushes of the mechanical commutator. However, the capacitor may only be used for selected types of noise environment, such as ultra-low noise environments.

RF traps 672 along the length of the shielded cable 664 can be used to prevent the shielded cable 664 from acting like an RF antenna and from absorbing and radiating RF energy around the Larmor frequency that is generated by the MR scanner. The RF traps 672 are used to suppress current from traveling on the shield of the wires 662. The shield currents, or common mode currents, can be suppressed by the RF traps. The RF traps are useful for patient safety as well. RF energy from the MRI scanner 650 can cause heating of long electrical wires that can result in patient burns. The RF traps 672 help to significantly reduce the possibility of RF energy heating the wires of the system. The use of shielded and grounded components, the capacitor 607 across the motor terminals (in some embodiments), the RF traps 672 along the shielded cable 664, and filtering of the motor controller currents, housing the motor in a conducting enclosure, and having a motor axle that is made from a material with low electrical conductivity are all aspects that enable this motor design to be operated simultaneous to MR imaging to achieve images whose quality are minimally affected by interference from the operation of the high external magnetic field compatible mechanically commutated electromagnetic motor 601.

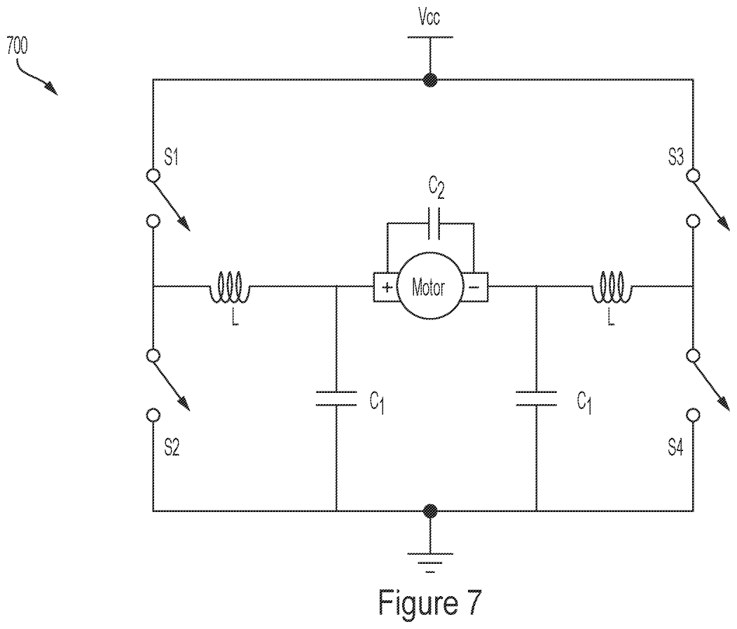

The H-bridge and the passive filtering circuit can be used in the motor controller 660 of FIG. 6. A circuit diagram is shown in FIG. 7 of a circuit schematic of an example H-bridge 700, filtering circuit, and motor. S1, S2, S3, and S4 denote the switches that comprise the H-bridge. An inductor-capacitor (LC) low-pass filter can be used on each output of the H-bridge. As was mentioned earlier, the H-bridge circuit can use PWM control schemes to vary the time-averaged amplitude of the current being supplied to the motor. The square waveforms of PWM contain a wide range of frequency components, many of which will overlap with the Larmor frequency of the MRI system. For 1.5 Tesla and 3 Tesla MRI systems, the Larmor frequency of protons is approximately 64 MHz and 128 MHz, respectively.

By adding an LC low-pass filter with a frequency cutoff below the Larmor frequency to the output of the H-bridge, any high frequency signals near the Larmor frequency can be substantially attenuated at the location of the motor controller. The inductor (L) and capacitor (C1) values can be chosen such that the low-pass filter cutoff frequency is sufficiently far from the Larmor frequency while only minimally impacting the controller signal output from the H-bridge. The C2 capacitor may be used in some embodiments to further reduce noise. Alternatively, the C2 capacitor may not be necessary in the H-bridge circuit. Here in FIG. 7 a low-pass filter on the output from the H-bridge is disclosed. However, other filter types such as a band-stop filter may also be used. As long as the production for RF noise near the Larmor frequency is minimized, any filtering scheme may be sufficient.

High Magnetic Field Compatible mechanically commutated Servo Motor

A servo motor is a motor that is configured to provide feedback information regarding the rotational position of the motor. Selected sensors can be used to provide feedback information, including optical sensors and Hall Effect sensors. A position encoder can send information obtained from the sensors to the controller to enable the controller to determine a position of the motor as the motor rotates. Optical sensors can, for example, detect physical markers on a rotor to provide position information.

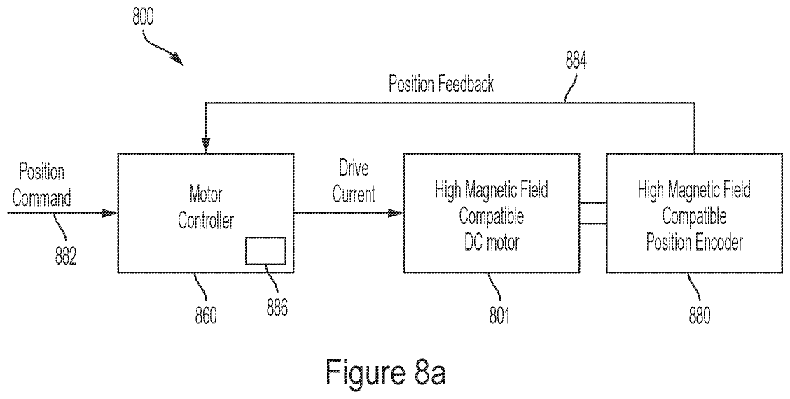

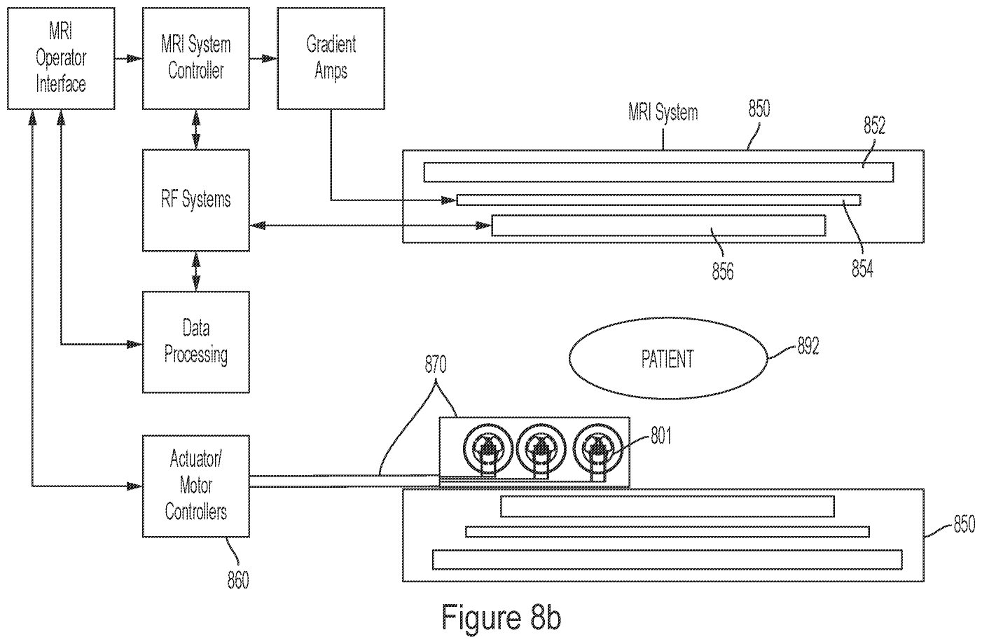

A high level schematic of an example of an external high magnetic field compatible mechanically commutated electromagnetic servo motor 800 is shown in FIG. 8a. In this example, the servo motor is configured to operate in an externally produced high magnetic field environment, such as an MR system. An external high magnetic field compatible position encoder 880 can be used to communicate the motor's rotor position back to the motor controller 860 using a non-magnetic communication means, such as a shielded cable or fiber optic cable. In one embodiment, the position encoder 880 can be coupled to an axle of the external high magnetic field compatible mechanically commutated electromagnetic servo motor 800.

An encoder, such as an optical encoder, can then be used to determine when each detector has passed a selected location. In addition, a single detector can also be configured to identify multiple transitions during a single rotation. For example, an optical detector may be configured to measure brightness or color. Selected threshold values can be used to identify multiple locations of the axle as it rotates through 360 degrees, thereby enabling a single detector to be used to identify multiple locations on the axle. The position encoder can be configured to measure and provide information regarding the position of the motor as it rotates. The measured position information of the motor from the position encoder can be fed back to a microcontroller 886, or other type of processor, in the motor controller 860. The microcontroller 886 can be configured to compare a desired position command 882 with a measured axle position that is fed back 884 to the microcontroller in the motor controller 860. In one example, a proportional-integral-derivative controller can be used to determine the desired switching scheme for the H-bridge 700 (FIG. 7) to achieve the desired position or number of revolutions of the motor rotor. As with the motor controller and motor design described in FIGS. 2 and 6, noise reduction strategies can also be used to minimize unwanted electromagnetic interference (EMI) from the signal containing the data that is sent from the motor 801 to the motor controller 860. This configuration allows for an external high magnetic field compatible mechanically commutated electromagnetic motor that offers closed loop control.

The high magnetic field compatible position encoder 880 illustrated in FIG. 8a can be of the incremental or absolute encoder type. Traditional magnetic encoding schemes are not possible due to the incompatibility of these encoders with the strong magnetic field of the MR system. Three different embodiments are disclosed for encoding the rotor position in a manner that can operate in an externally produced high magnetic field environment: (1) a fiber-optic system that uses both optical detection of the rotor position and uses fiber optic cables to transmit the encoded position back to the motor controller 860. The high magnetic field compatible position encoder 880 can transmit a digital signal representative of the position of the rotor to the motor controller 860. This configuration ensures that transmission of rotor position is unaffected by radio frequency noised generated by another electronic system, such as an MRI system. (2) The use of Hall Effect sensors and corresponding circuitry and wiring to transmit an electrical signal back to the motor controller 860 that details the position of the rotor relative to the main magnetic field 240 of the MRI system 250 (FIG. 2a). (3) An optical encoding scheme that transmits information via electrical wires instead of via a fiber optic cable. This approach can include an encoding disk having clear and opaque sections being attached to the motor output. In one example, one or more light sources can be used to illuminate one side of the disk and light sensors and corresponding circuitry can be used to detect light signals transmitted through the disk. These electrical signals can be communicated to the motor control unit to provide feedback. Method (2) and (3) use a wired connection from the servo motor area 801 back to the motor controller 860. As was described for the wires 662 going to the positive and negative leads of the motor 601 (FIG. 6), any additional communication wires can be properly shielded cables 664 with RF traps 762 to ensure transmitted signals are substantially clean from noise generated by the MR system 650, and also don't contribute noise to the MR system 650 (FIG. 6). Alternatively, a fiber optic cable can be used to send the position feedback 884 from the high magnetic field compatible position encoder 880 to the motor controller 860. Two or more of these encoding schemes can be used together. For example, method (2) can be used with (1) or (3) to know both the rotor position and the position of the rotor relative to the main magnetic field of the MRI.

FIG. 8b presents a high level schematic of multiple high magnetic field compatible mechanically commutated electromagnetic servo motors 801 and controllers integrated with the MRI system 850. The multiple high magnetic field compatible mechanically commutated electromagnetic servo motors 801 are illustrated as located within the bore of the MRI system 850. The high field magnet 852, gradient coil 854, and body coil 856 are illustrated, as in FIG. 2a. The servo motors 801 are surrounded by RF shielding 870. The multiple servo motors can each be used to operate a different segment of a robotic system to operate on the patient 892 while the MRI system 850 is operating in real time. A motor controller 860 can be located outside of the MRI system 850 and connected with the servomotors 801 using shielded cables, as previously discussed. An MRI operator interface can enable an operator to control the MRI system 850 and the high magnetic field compatible mechanically commutated electromagnetic servo motors 801 via the motor controllers 860, and an MRI system controller that is used to control gradient currents, RF systems, and data processing of the MRI system. The use of the RF shielding 870 and the high magnetic field compatible mechanically commutated electromagnetic servo motors 801 within the bore with the high magnetic field strength of the high field magnet enables images to be produced by the MRI system 850 with little effect on the SNR of the MR images.

### MRI-Compatible Electromagnetic Servomotor and Performance

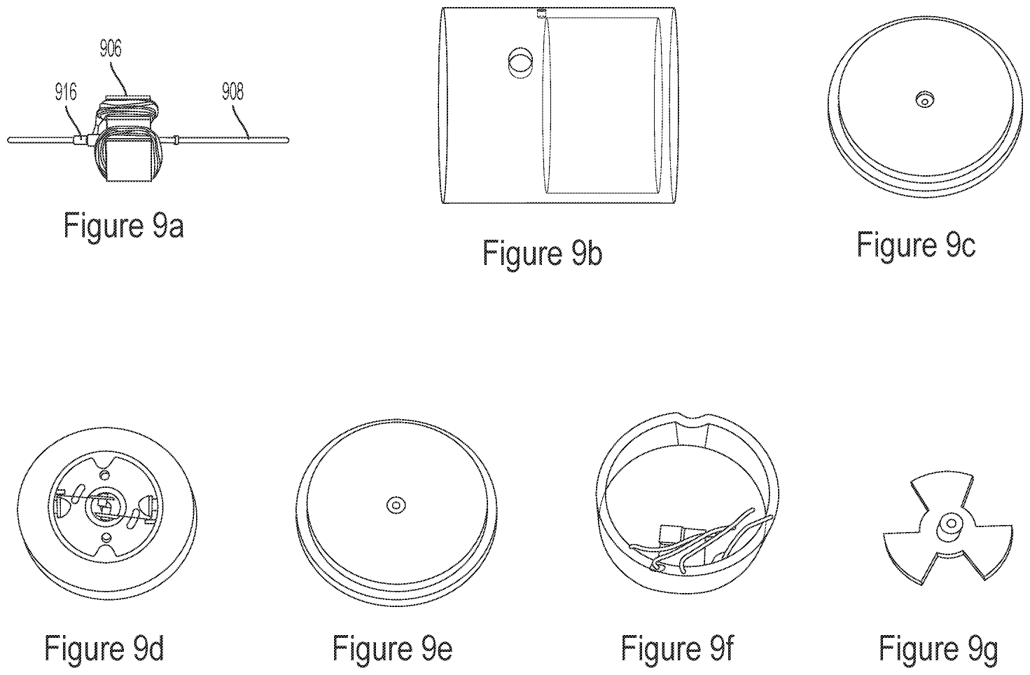

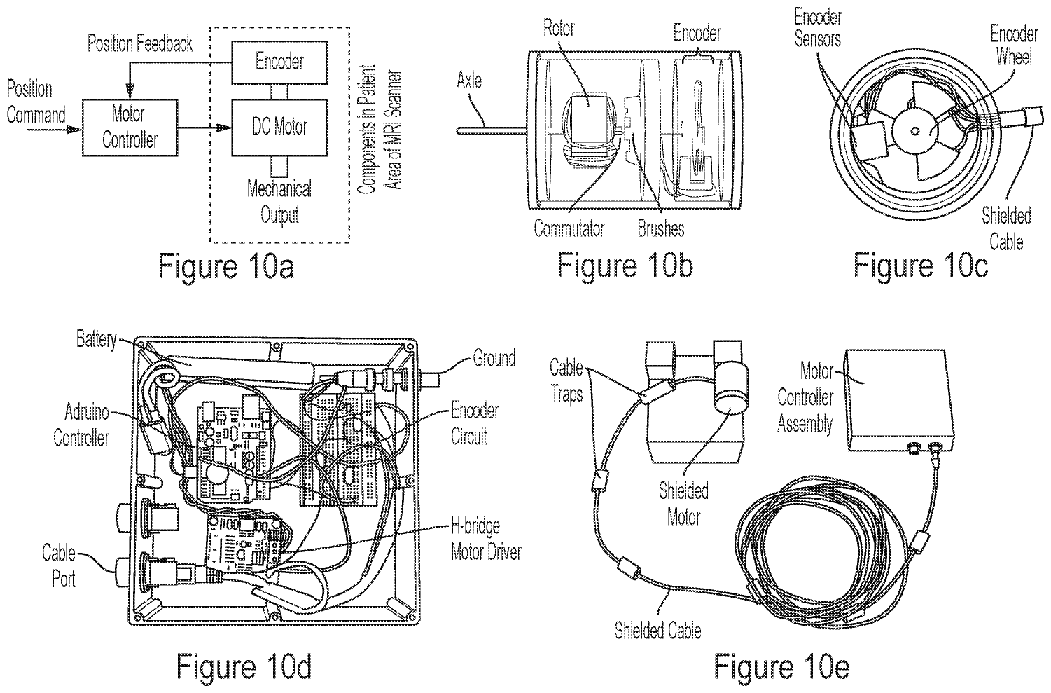

In one example, the external high magnetic field compatible mechanically actuated motor concept in FIG. 2a can be combined with a non-magnetic optical encoder and motor controller, as illustrated in FIGS. 9a to 10e to achieve closed loop high magnetic field compatible mechanically actuated servomotor functionality in an external high magnetic field environment, such as an MRI system.

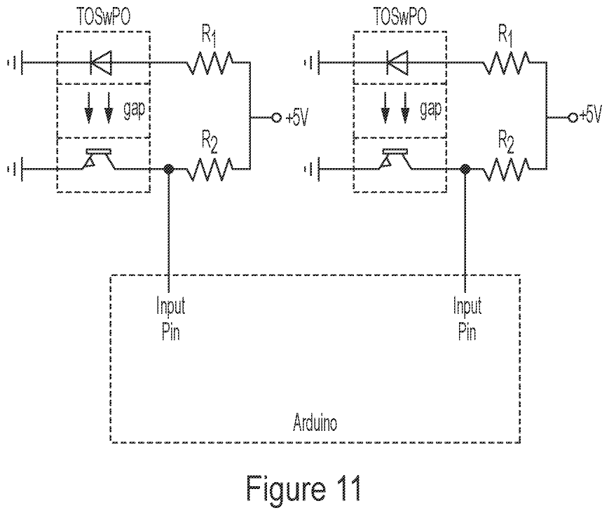

FIGS. 9a through 9g show external high magnetic field compatible mechanically actuated servomotor (servomotor) components prior to assembly. The assembled servomotor is shown in FIG. 10b. An end view of the servomotor shown with the encoder assembly is illustrated in FIG. 10c. A component of the external high magnetic field compatible mechanically actuated motor is illustrated in FIG. 9a, with the commutator 916 and nonmagnetic rotor 906 attached to the low-conductivity axle 908. A polycarbonate housing, FIG. 9b, is configured to encase the servomotor, with a housing end shown in FIG. 9c. A first end ring, shown in FIG. 9e can be carried on a first side of the axle 908. A second end ring with brushes, shown in FIG. 9d, can be carried on a second side of the axle 908 with the brushes in communication with the commutator. The encoder in this example comprises two transmissive optical sensors, shown in FIG. 9f, with transmissive optical sensor with phototransistor output (TOSwPO) units that can detect changes in position and direction of a three leaf encoder disk, shown in FIG. 9g, that rotates when it is attached to the motor axle 908. In this example, the encoder is configured to subdivide each axle revolution into 12 increments. Fewer or greater number of increments may be used to obtain a desired amount of information regarding the rotation of the axle 908. The encoder circuit used to detect changes in the TOSwPO sensors is in the upper right of FIG. 10d with the circuit diagram shown in FIG. 11. The encoder sensors, encoder wheel, and shielded cable are shown partially assembled in FIG. 10c.

As shown in the block diagram of FIG. 10a, a position command can be sent to the motor controller, which then sends a signal to the servomotor connected to the encoder. The encoder then measures the position of the axle and sends position feedback to the motor controller. Control of the rotor position may be achieved using a proportional integral derivative (PID) controller or other feedback control strategy implemented on a microcontroller (such as an Arduino microcontroller) (FIG. 10d) which receives inputs from the encoder circuit. The speed, acceleration, and directional control of the motor can be achieved in one example embodiment using an H-bridge controller (in FIG. 10d) which sends a pulse width modulation (PWM) control signal to the servomotor. The servomotor is illustrated in FIG. 10b, assembled within the non-magnetic housing illustrated in FIG. 9b, which is capped with the housing end shown in FIG. 9c. A shielded Cat7 Ethernet cable electrically connects the servomotor to the controller unit at the cable port, as shown in FIG. 10e. A battery is used to power the electronics on the Arduino controller, the H-bridge motor driver, and the encoder circuit. A ground connection to a system can be made to the motor control assembly of FIG. 10d. The final motor control and servomotor assembly including all electromagnetic interference (EMI) shielding and radio frequency (RF) cable traps is shown in FIG. 10e.

In accordance with one embodiment, connecting a battery, such as a lithium polymer battery, to the DC motor terminals may be used to induce a current in the coils that can be phased to create an electromagnetic field that provides rotary motion of the motor rotor and axle relative to the magnetic field of the superconducting magnet of the MRI system, as previously discussed. The motor operation can be achieved at different orientation angles in the high ambient magnetic field environment, such as the environment within the MRI bore. Thus, precise control of an electromagnetic servomotor constructed completely from non-magnetic components and operated inside the MRI scanner bore was demonstrated using conventional servomotor control principles.

In one example embodiment, servomotor performance metrics when powered by a 7.4 V lithium polymer (LiPo) battery and operated at field strength of 2.89 Tesla (T) are shown in Table 1. Stall torque and unloaded shaft speed are sufficient for many actuation applications. Servomotor diameter and length in this example are 58, and 74 mm, respectively. Different diameters and lengths can be selected based on the system design to achieve a desired stall torque and speed.

TABLE 1
Servomotor performance measurements while operating
in MRI-system with a field strength of 2.89 Tesla.

| | No Load | Stall |
|---|---|---|
| Current (A) | 0.07 | 1.58 |
| Voltage (V) | 8.29 | 7.65 |
| Speed (rpm) | 1524 | — |
| Torque (mNm) | — | 73.1 |

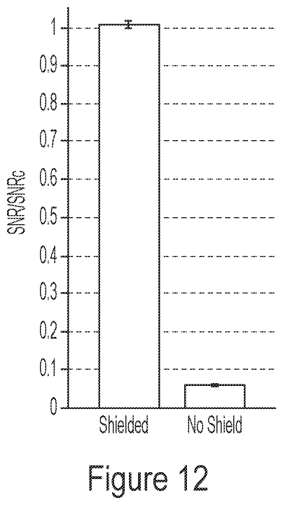

As previously discussed, the MRI transmit/receive hardware is extremely sensitive to RF energy. Sources of electromagnetic noise near the proton Larmor frequency (123.23 MHz @ 2.89 T) can significantly degrade imaging performance by introducing unwanted electromagnetic signal into the image receiver hardware. The making and breaking of electrical contacts between the brushes and stator during servomotor operation generates broadband radio frequency (RF) noise over a wide frequency band and this noise source can degrade MRI image quality if not sufficiently corrected for. FIG. 12 shows the signal to noise ratio (SNR) for a shielded motor and an unshielded motor. The SNR of the shielded motor is approximately 20 times greater than the unshielded motor. The H-bridge motor controller uses pulse width modulation (PWM) to control the effective voltage signal to the motor leads. The square voltage waveforms generate broadband RF noise which is a potential noise source to consider.

To minimize interactions between the high magnetic field compatible mechanically actuated servomotor and the MRI system, three critical design aspects were incorporated into the servomotor and controller shown in the example illustration of FIG. 10e. First, EMI shielding was used to prevent broadband energy produced by the H-bridge controller and motor brushes from radiating to the MRI receiver hardware. The servomotor was housed in a continuous copper shield (FIG. 10e). A 2 mm hole in one end of the shield allowed the motor axle to penetrate the housing. The motor controller unit and associated electronics were enclosed in a grounded and shielded box. Power and control signals between motor and motor controller unit can be transmitted by a double shielded Cat7 Ethernet cable (4 twisted pairs, one pair supplies current to run the motor, one pair is used to power the encoder diodes, and two pairs are used to return sensor signals to the motor controller). The shield of the Cat7 cable was soldered to the motor shield and electrically connected to the grounded shielded box of the motor controller using associated RJ45 connectors (FIG. 10e). Second, to prevent the motor axle from acting as an antenna and radiating noise contained inside the motor faraday cage, a low conductivity Garolite G-10/FR4 2 mm composite axle (Mcmaster Carr) was used. Third, six cable traps tuned to the proton Larmor frequency were installed and spaced 15 cm apart on both ends of the shielded cable to attenuate any RF energy near 123.23 MHz from traveling on the cable shield. These cable traps serve two important functions: (1) to prevent unwanted common mode currents at the Larmor Frequency from entering the patient area of the MRI system and (2) to minimize potential heating of the shielded cable by the MRI transmit magnetic field. The components listed in this embodiment are exemplary only and are not intended to be limiting in any way.

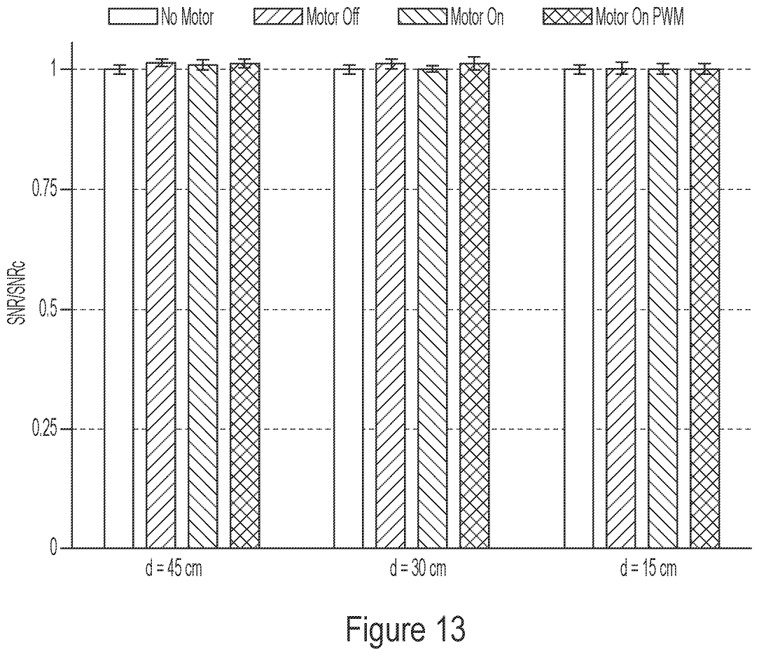

Results illustrated in FIG. 13 demonstrate that the use of the EMI design strategies listed above limits unwanted interactions between an MRI system and the operating servomotor. The measured signal to noise ratio (SNR) of images acquired using the MRI differed from a control image (SNRc) by no more than 1.5% for a range of test configurations during motor operation. The motor position was varied between 45 and 15 cm and the servomotor was controlled with an H-bridge motor controller and powered by a DC voltage supply. FIG. 13 shows that for all test conditions and distances, the measured image signal to noise ratio was remarkably similar to imaging in the absence of the servomotor unit. Thus, the ability to simultaneously image with an MRI and operate an external high magnetic field compatible mechanically actuated servomotor using conventional actuation principles and motor controllers was demonstrated. This capability may unlock the full potential of robotic assisted procedures performed under real-time imaging guidance with intraoperative MRI.

### ACTUATOR DIAGNOSTIC APPLICATION EXAMPLES

### Example 1

The external high magnetic field compatible mechanically commutated electromagnetic motor, as described herein, can be configured to be used in a number of different uses within an external high magnetic field environment.

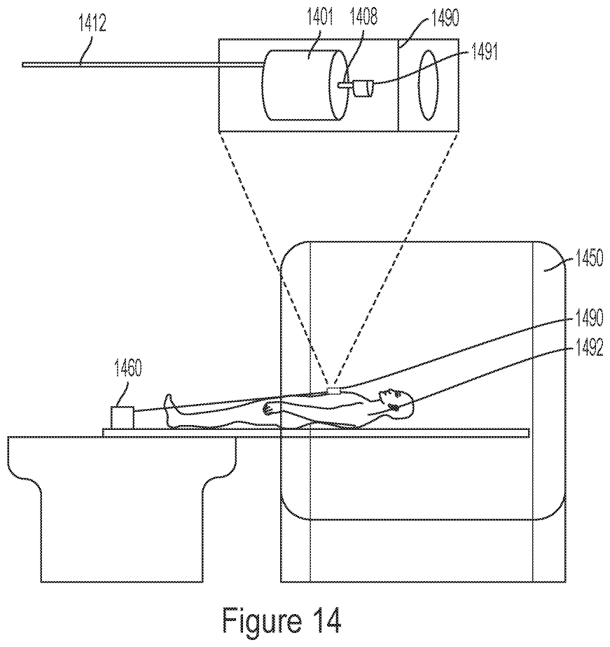

One example use is illustrated in FIG. 14. An external high magnetic field compatible mechanically commutated electromagnetic servomotor 1401, configured for operation within an MR system 1450, is illustrated with wire leads coupled to terminals 1412 and an unbalanced weight 1491 attached to the motor axle 1408 to form a mechanical excitation source device 1490. The mechanical excitation source device can be coupled to a servomotor control unit 1460 that is configured to operate the servomotor at a desired speed. The mechanical excitation source device 1490 can be used as a harmonic driver to enable the servomotor to be used for magnetic resonance elastography (MRE). This driver is configured to provide mechanical excitations to tissues of a patient 1492 at a selected mechanical frequency to generate a propagating wave in the tissues. The tissues can then be imaged in real time by the MR system while the propagating wave is traveling through the tissues.

When the mechanical excitation source device 1490 is powered to produce rotary motion, the motor unit vibrates. The servomotor 1401 is connected back to a servomotor control unit 1460 which, using feedback from an axle encoder, can precisely control the revolutions per minute of the motor and hence the harmonic excitation frequency of the MRE driver. This driver can be controlled to operate at 60 Hz, 100 Hz or other frequencies of interest to provide mechanical excitations to the tissue. This harmonic excitation may be timed with the gradient waveforms and imaging protocols of the MRI scanner to achieve the desired elastography measurement. The mechanical excitation device 1490 can be operated simultaneous to imaging by the MR system 1450.

One significant benefit of this driver over existing MRE driver technology is that it is simple, low cost, and yet can be controlled to produce a wide range of mechanical excitation frequencies and forcing functions. Current clinically used MRE driver systems involve extensive hardware that is located outside an MRI control room. This hardware is expensive and difficult to integrate with an MRI system in the hospital, which is likely to limit or slow the wide adoption of MRE. Another challenge with the clinically used MRE drivers is that they are typically air-powered. Air is not an ideal hydraulic medium and its compressibility limits the range of driving frequencies and force profiles that can be achieved.

In another embodiment the MRI compatible DC motor unit 1401 can also be used as a generator in the MRI scanner 1450 to measure mechanical output from the patient 1492 during or immediately before or after imaging. The mechanical output can be determined by applying a known torque to a mechanical arm attached to the external high magnetic field compatible mechanically commutated electromagnetic servomotor 1401. A patient can push against the torque. The position of the mechanical arm can be determined using the motor controller. The torque can be increased to keep the mechanical arm within a certain location as the patient attempt to move it. The amount of torque needed to keep the mechanical arm within the desired location can be used to determine how much force the patient can apply.

In another embodiment, pedals can be attached to the MRI compatible DC motor unit 1401 and the motor unit may be connected to an electrical load. The pedals can be formed using a non-magnetic material to enable a patient to provide mechanical output to the pedals while patient and pedals are within an MRI machine. A patient can rotate the pedals which are coupled to the motor unit 1401, thereby generating a current in the motor and load to produce a selected amount of power. The power generated in the MRI compatible DC motor unit 1401 can be used to determine how much work was performed by the patient. The work performed by the patient can be used as a type of stress test. Such a stress test could be useful for cardiac imaging or other protocols. The power generated by the generator can be used to measure physiological output produced by the patient 1492 while in the MRI scanner 1450 during exercise.

### Example 2

Combining the precision of robotic-assisted procedures with the soft-tissue imaging capabilities of intraoperative magnetic resonance imaging (MRI) has the potential reduce invasiveness and improve precision in a variety of surgical settings. However, electromagnetic actuators that have been widely vetted and used for commercial medical robotics are inherently incompatible with MRI—magnetic actuator components can become dangerous projectile near strong magnetic fields. In this example, an electromagnetic servomotor that is made from non-magnetic materials and uses the magnetic field of the superconducting magnet of the MRI scanner (Bo field) for actuation. Electrical currents supplied to rotor windings in the servomotor create an electromagnet that can interact with the magnetic field to produce high torque rotary actuation. An optical encoder that detects motion of the servomotor axle can be wired to a remote motor controller to enable closed-loop control. Simultaneous servomotor operation and artifact-free MRI is achieved by enclosing the servomotor in a faraday cage, constructing the servomotor axle from non-conducting materials to prevent radio frequency (RF) noise from escaping the faraday cage, and using resonant RF traps on cabling to the servomotor, as previously disclosed. Using this MRI-compatible servomotor design, a proof-of-concept surgical robot for biopsy needle placement under real-time image guidance was built. An MRI operating at 5 frames/second was used to track a biopsy introducer needle movement during continuous robot operation. Robotic placement of a 9-gauge introducer sheath under MRI-guidance was performed to gain access to a desired biopsy target. Thus, demonstrating that widely used electromagnetic actuation principles can be safely used in medical robots designed to operate under real-time image guidance with MRI.

In this example a servomotor is constructed from non-magnetic materials and yet unlocks the paradigm of utilizing electromagnetic actuation in close proximity to the superconducting magnetic field of the MRI system. This actuator design can be operated simultaneous to the MRI without degrading image quality. An optical rotary encoder and servomotor controller enable closed-loop control of the servomotor. An MRI-compatible surgical robot using this electromagnetic servomotor actuator can be used to drive a biopsy introducer to the desired target of interest while imaging at 5 frames/second.

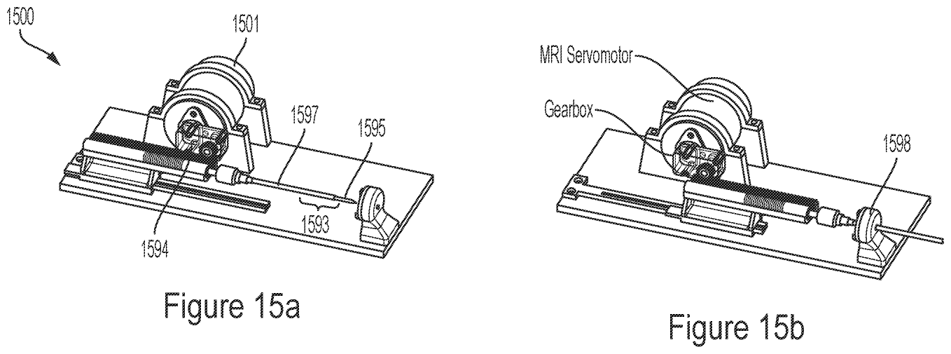

FIG. 15a illustrates a biopsy introducer robot 1500 actuated by an MRI-compatible electromagnetic servomotor 1501. FIG. 15a illustrates an example of a single degree-of-freedom biopsy introducer robot prior to introducer insertion. The servomotor 1501 is an external high magnetic field compatible mechanically commutated electromagnetic motor that is coupled to an introducer 1593 via a gearbox 1594. In one example, the gearing connecting an output of the servomotor 1501 to the linear stage results in a maximum linear stage speed of approximately 10 millimeters per second (mm/s) and a maximum insertion force of about 131 pounds (585 Newtons). The total range of the linear stage travel is about 10 cm.

The servomotor 1501 can provide feedback to enable a controller to accurately determine how far an introducer sheath 1597 is inserted through a sheath holder 1598 to direct the cutting stylet 1595 to a desired location within an external high magnetic field environment, such as the bore of an MR system. FIG. 15b shows the introducer and cutting stylet at the maximum insertion depth. The sheath holder 1598 allows the position of the introducer sheath 1597 to be maintained during removal of cutting stylet 1595. The biopsy introducer robot can include a Vernier scale on a linear slide with 0.1 mm increments. This Vernier scale can be used for system calibration when the robot is first powered on. The tip of the cutting stylet can be imaged with an MRI to determine its location within a subject in the MRI. The servomotor 1501 can advance the introducer in a controlled manner during continuous imaging at 5 frames per second. These results constitute an important step towards highly functional robotic systems that can be used to perform surgical procedures under concurrent intraoperative MRI guidance.

In one example, the biopsy introducer robot 1500 was configured with a 9-gauge biopsy introducer sheath under real-time MRI-guidance. An illustration of the 1-degree-of-freedom robot is shown in FIG. 15a-b where FIG. 15a shows introducer in a retracted position, and FIG. 15b shows the introducer in the fully inserted position. The constructed robot is shown in FIG. 15a. A Vernier scale on the introducer stage (not shown) can be used to calibrate the position of the linear stage controlling introducer placement. Gearing connecting servomotor output to linear stage results in a maximum linear stage speed of 10 mm/s and a maximum insertion force of 131 pounds (lbs.) (585 Newtons (N)). Total range of linear stage travel is 10 centimeters (cm).

### Example 3

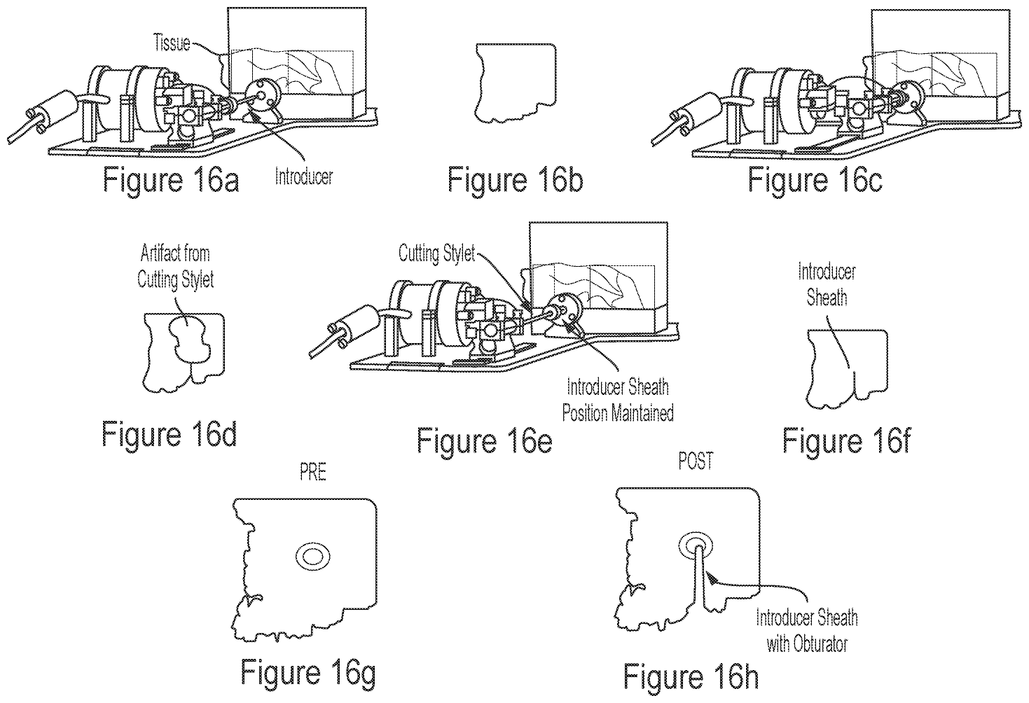

The MRI-compatible biopsy insertion robot 1500 was then used to place a 9-gauge introducer sheath to a pre-determined tissue target during continuous MR imaging, as shown in FIGS. 16a-h. Volumetric MRI was performed prior to needle insertion to determine a desired introducer sheath placement location in imaging coordinates, as shown in FIG. 16g. The robot was commanded under one continuous operation to drive the cutting stylet and introducer sheath from an initial position (FIG. 16a) to a maximum insertion depth (FIG. 16c) and then to remove the cutting stylet from the introducer sheath (FIG. 16e). The corresponding real-time images for each of these steps are shown in FIGS. 16b, 16d and 16f with the pre and post introducer sheath placement images shown in FIGS. 16g and 16h. This ex vivo tissue experiment demonstrates that a proof-of-concept surgical robot powered by the MRI-compatible electromagnetic servomotor can drive a large bore introducer through tissue to reach a desired and predetermined target.

Servomotor Construction Details

Components of a prototype servomotor are shown in FIGS. 9a-g. The prototype provides one example of a servomotor that is configured for use in a high external magnetic environment. The motor axle 908 was constructed from a 2 mm diameter G-10/FR4 non-conducting rod (McMaster-Carr, #8669K627). Mechanical commutator and brushes were obtained from a disassembled 280 micro 3 volt-12 volt DC toy motor. The rotor 906 support structure for the rotor windings was 3D printed from VeroWhitePlus (Stratasys, Israel). Each of three 100-turn rotor windings (˜20 mm² cross-sectional area) was hand wound from 30 gauge Polyamideimide magnetic wire (Remington Industries, Illinois, USA). Once wound, cyanoacrylate glue was used to secure rotor windings in place. Solder was used to connect rotor windings to commutator. The measured resistance of each rotor loop was 1.2Ω.

The outer housing of the servomotor was constructed from outer motor housing was constructed from 2¼″ outer diameter clear polycarbonate tubing shown in FIG. 9b (McMaster-Carr, #8585K28) Motor end rings in FIGS. 9d and 9e were 3D printing from ABS plastic and 2 mm inner diameter Olite bushing (McMaster-Carr, #6658K411) were pressed into the motor end rings and provided support for the motor axle 908. Powdered graphite lubricant (Panef Corp. Milwaukee, WI) was used to minimize friction between axle 908 and bushings. One end ring, FIG. 9d, had 3D printed details to enable proper alignment and fixation of the brushes to the end ring. A 2″ outer diameter clear polycarbonate tubing, FIG. 9b, (McMaster-Carr, #85851(26) was used to maintain alignment and proper separation of motor end rings. Two additional tight-fitting bushings (not shown in FIGS. 9a-g) were secured to the axle to keep the rotor properly situated between the two end rings of the motor housing.

The encoder, FIG. 9f was constructed from a 3D printed ABS plastic encoder wheel (5 mm thickness, 35 mm maximum diameter). To ensure opacity of encoder wheel, FIG. 9g, each leaf was spray coated with black paint. The two TOSwPO sensors illustrated in FIG. 9f (TCST2103, Vishay Intertechnology Inc.) were mounted to the circular support polycarbonate tubing (2″ outer diameter) so that four unique states of the sensors were possible: (1) both sensors blocked by an opaque encoder leaf, (2) first sensor blocked by an opaque encoder leaf and second not blocked, (3) second blocked by an opaque encoder leaf and first not blocked, (4) neither sensor blocked. These four unique states enabled rotor motion and direction to be measured.

The constructed servomotor assembly without EMI shielding is shown in FIGS. 10b and 10c. To install the EMI shielding, the outer polycarbonate housing of the servomotor was coated in copper shielding foil tape (3M, #1739-17), as shown in FIG. 10e. Tape seams were soldered to ensure electrical connection between all segments. A 25-foot double-shield Cat7 Ethernet cable, shown in FIG. 10e, consisting of 4-twisted pair 26 gauge wires (Tera Grand, California) was used to transmit all signals between the shielded servomotor (FIG. 10b) and the motor control assembly (FIG. 10e). One twisted pair was used to supply current to motor terminals. A second twisted pair was used to supply power to the diodes on the TOSwPO sensors. The remaining two twisted pairs communicate the TOSwPO sensor signals to the motor controller assembly. All electrical connections at the servomotor were soldered and shield of Cat7 cable was soldered to copper tape on the motor housing. An RJ45 connector on the end of the Cat7 cable distant from the servomotor allows easy connection of wires and cable shield to the motor controller assembly.

The motor controller assembly (FIG. 10d) was housed in an 18.8×18.8×6.7 cm aluminum box. A female RJ45 connecter (PEI-genesis, PA, USA) enabled easy connection of the servomotor to the motor controller assembly. The shielded box, cable shield, and motor shield are all connected to ground via a BNC connector on the back of the box (FIG. 10d). All power to servomotor and controller is provided by a 7.4V 2-cell LiPo 2100 milli-amp-hour (mAh) battery (Hobbyking, Hong Kong). All control logic was implemented on the Arduino Uno Rev3. The TOSwPO sensor signals are fed as inputs to the Arduino which then sends the desired PMW control signal to a 2-amp H-bridge motor controller (DFRobot, DRI00002). Additional circuit (see circuit diagram in FIG. 11) used to read TOSwPO signals is located on a solderless breadboard (see FIG. 10d).

Six floating shield current suppression traps (39) were constructed and installed 15-cm apart (FIG. 10e) on the terminal ends of the Cat7 ethernet cable in order to suppress common mode currents on the cable shield. RF traps were constructed to attenuate signals at 123.23 MHz (the proton Larmor frequency at 2.89 Tesla) to further minimize interactions between MRI transmit/receive hardware and servomotor hardware. Outer and inner diameter of traps were 22 mm and 7 mm, respectively. Two trap variants with a length of 38 mm and 57 mm had a mean attenuation at 123.23 MHz of 7.4 dB and 11.3 dB, respectively.

Encoder Sensing and Controller

Accurate detection of the signal from the TOSwPO sensors can provide precise servomotor control. The schematic in FIG. 11 show the circuit used to power the TOSwPO diodes as well as the voltage dividing sensing circuit. The voltage at the Arduino input pin for a given sensor is low when the opaque encoder wheel leaf is not blocking the transistor sensor, otherwise the voltage is high. Control software was implemented on the Arduino to continuously monitor the state of the sensor signal to allow changes in rotor position to be detected and the number of rotation increments to be counted. The Arduino was programmed to receive a commanded input (in number of encoder steps) from the user. A proportional integral (PI) controller was then use to generate a control signal sent to the H-bridge motor controller to achieve the desired commanded actuation.

Servomotor Performance Measurements

Stall torque and unloaded motor speed was measured for the servomotor operating in the 2.89 T magnetic field of a clinical MRI scanner. For both measurements, motor was directly powered by the 7.4V LiPo battery. A Fluke 77 and Fluke 27 multimeter were used to measure voltage across the motor leads and rotor current during operation. A mass was attached to the 2-inch diameter pully mounted on the servomotor axle. The amount of mass was increased incrementally until the maximum lifting capacity of the motor-pully assembly was determined. The reported stall torque is the product of the maximum lifted weight times the pully radius. To determine the maximum motor shaft speed, the servomotor was powered in the unloaded state. The encoder hardware and associated circuitry was used to count the number of full axle revolutions occurring over a one minute interval.

SNR Measurement During Motor Operation

Interactions between the operating servomotor and MRI were evaluated using signal to noise (SNR) measurements. Simultaneous imaging and motor operation was tested for three different states: (1) Motor off but located in the MRI scanner bore, (2) Motor on and powered by the 7.4V LiPo battery, and (3) Motor on and powered by the PWM signal output from H-bridge motor controller. The distinction between (2) and (3) was that the PWM signal from the motor controller generated at 50% duty cycle square wave voltage signal. For each test condition, image quality of the MRI was evaluated by measuring the signal to noise ratio (SNR) for each acquired image. SNR measurement were obtained for three different motor-phantom separation distances (d=15, 30, 45 cm), as illustrated in FIG. 13. For each separation distance, the three motor states (described above) were tested. A control SNR measurement (no motor) was also acquired with motor and motor controller completely removed from the MRI scanner room.

For SNR measurements, a 2 channel transmit/receive body coil was used to acquire a 2D gradient echo image (GRE) image in the coronal plane with the following acquisition parameters was used: echo time/repetition time (TE/TR)=3.58/200 ms, flip angle=60°, field of view (FOV)=22 cm, resolution=1.72×1.72×5 mm, bandwidth=260 Hz/pixel, 1 averages. Images for each coil were reconstructed from raw k-space data in MATLAB (MathWorks, Natick, MA). SNR maps were formed using the noise-covariance-weighted sum of squares magnitude image reconstruction method. Noise covariance information was calculated from pixels in the over-scan area of the image that was dominated by noise. For each scenario, 12 independent SNR maps were acquired. For each map, the mean SNR over the phantom cross-section was calculated and reported as a single SNR image quality metric. The mean and standard deviation of this mean SNR image quality metric was calculated and reported for each tested scenario.

Biopsy Introducer Robot Construction

A proof-of-concept one degree of freedom robot was constructed from non-magnetic components (FIG. 15a-b). A modified plastic Vernier caliper was used as a linear stage and the 0.1 mm scaling was used for initial calibration of the linear stage position when the robot was first powered on. The MRI-compatible electromagnetic servomotor described earlier in this paper was used for actuation. A 120:1 Plastic Gearmotor (Popolu) was mounted to the output axle of the servomotor using a 3D printed gearbox holder made from ABS plastic (shown in FIGS. 15a and 15b). To ensure that the gearmotor was non-magnetic, the ferromagnetic steel axles in the gearbox were replaced by 2 mm diameter 316-stainless steel axles (McMaster Carr, #TKTK). A 15 tooth 15 mm diameter plastic gear (McMaster-Carr, 2262N415) was attached to the output of the gearmotor and coupled to a matching linear gear rack (McMaster-Carr, 266N57) to provide actuation of the linear stage. A 3D printed sheath holder allows the biopsy introducer sheath 1597 to be held in a fixed position during removal of the cutting stylet 1595. To demonstrate simultaneous imaging and actuation of the robot, a mock introducer consisting of a fiberglass rod with cylindrical fiducial marker was constructed.

An ex vivo tissue experiment was preformed to demonstrated accurate placement of a 9-gauge introducer into a pre-specified target during simultaneous imaging with MRI. The introducer 1593 which is comprised of a cutting stylet 1595 and introducer sheath 1597 used for MRI-guided breast biopsy procedures (Hologic), was mounted onto the robot linear stage as is shown in FIG. 15a. A sheath holder 1598 (shown in FIG. 15b) that uses a rubber friction mechanism was built to both allow the insertion of the introducer 1593 and to hold the introducer sheath 1597 at the desired insertion depth during removal of the cutting stylet 1595.

Rapid MRI Imaging Protocols used During Robot Actuation

Single slice 2D MRI was used to track the mock introducer tip location during the phantom experiment and to actively monitor the introducer insertion during the ex-vivo experiment. The spine coil array mounted in the patient table was used. Pulse sequence parameters were chosen to achieve an imaging rate of 5 frames/second (0.2 seconds per image). For the phantom experiment, a TRUFI pulse sequence was used with the following scan parameters: TE/TR=1.94/3.87 ms, flip angle=45°, matrix=256×62, resolution=1.17×1.25×5 mm, bandwidth=1149 Hz/pixel, partial Fourier in phase-encoding=5/8, GRAPPA parallel imaging with 22 reference lines and an acceleration factor of 2. For the ex vivo experiment, a FLASH pulse sequence was used with the following scan parameters: TE/TR=2.24/4.9 ms, flip angle=8°, matrix=256×58, resolution=1.17×1.46×5 mm, bandwidth=1150 Hz/pixel, partial Fourier in phase-encoding=6/8, GRAPPA parallel imaging with 24 reference lines and an acceleration factor of 2.

The actuator technology described above is an advancement that has the potential to have a significant impact on MRI-guided interventions and the construction of cheap, simple, and effective actuators in the MRI environment.

Various techniques, or certain aspects or portions thereof, can take the form of program code (i.e., instructions) embodied in tangible media, such as floppy diskettes, compact disc-read-only memory (CD-ROMs), hard drives, non-transitory computer readable storage medium, or any other machine-readable storage medium wherein, when the program code is loaded into and executed by a machine, such as a computer, the machine becomes an apparatus for practicing the various techniques. Circuitry can include hardware, firmware, program code, executable code, computer instructions, and/or software. A non-transitory computer readable storage medium can be a computer readable storage medium that does not include signal. In the case of program code execution on programmable computers, the computing device can include a processor, a storage medium readable by the processor (including volatile and non-volatile memory and/or storage elements), at least one input device, and at least one output device. The volatile and non-volatile memory and/or storage elements can be a random-access memory (RAM), erasable programmable read only memory (EPROM), flash drive, optical drive, magnetic hard drive, solid state drive, or other medium for storing electronic data. One or more programs that can implement or utilize the various techniques described herein can use an application programming interface (API), reusable controls, and the like. Such programs can be implemented in a high level procedural or object oriented programming language to communicate with a computer system. However, the program(s) can be implemented in assembly or machine language, if desired. In any case, the language can be a compiled or interpreted language, and combined with hardware implementations.

As used herein, the term processor can include general purpose processors, specialized processors such as VLSI, FPGAs, or other types of specialized processors, as well as base band processors used in transceivers to send, receive, and process wireless communications.

It should be understood that many of the functional units described in this specification have been labeled as modules, in order to more particularly emphasize their implementation independence. For example, a module can be implemented as a hardware circuit comprising custom very-large-scale integration (VLSI) circuits or gate arrays, off-the-shelf semiconductors such as logic chips, transistors, or other discrete components. A module can also be implemented in programmable hardware devices such as field programmable gate arrays, programmable array logic, programmable logic devices or the like.

In one example, multiple hardware circuits or multiple processors can be used to implement the functional units described in this specification. For example, a first hardware circuit or a first processor can be used to perform processing operations and a second hardware circuit or a second processor (e.g., a transceiver or a baseband processor) can be used to communicate with other entities. The first hardware circuit and the second hardware circuit can be incorporated into a single hardware circuit, or alternatively, the first hardware circuit and the second hardware circuit can be separate hardware circuits.

Modules can also be implemented in software for execution by various types of processors. An identified module of executable code can, for instance, comprise one or more physical or logical blocks of computer instructions, which can, for instance, be organized as an object, procedure, or function. Nevertheless, the executables of an identified module need not be physically located together, but can comprise disparate instructions stored in different locations which, when joined logically together, comprise the module and achieve the stated purpose for the module.

Indeed, a module of executable code can be a single instruction, or many instructions, and can even be distributed over several different code segments, among different programs, and across several memory devices. Similarly, operational data can be identified and illustrated herein within modules, and can be embodied in any suitable form and organized within any suitable type of data structure. The operational data can be collected as a single data set, or can be distributed over different locations including over different storage devices, and can exist, at least partially, merely as electronic signals on a system or network. The modules can be passive or active, including agents operable to perform desired functions.

Reference throughout this specification to “an example” or “exemplary” means that a particular feature, structure, or characteristic described in connection with the example is included in at least one embodiment of the present invention. Thus, appearances of the phrases “in an example” or the word “exemplary” in various places throughout this specification are not necessarily all referring to the same embodiment.

As used herein, a plurality of items, structural elements, compositional elements, and/or materials can be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. In addition, various embodiments and example of the present invention can be referred to herein along with alternatives for the various components thereof. It is understood that such embodiments, examples, and alternatives are not to be construed as de facto equivalents of one another, but are to be considered as separate and autonomous representations of the present invention.

Furthermore, the described features, structures, or characteristics can be combined in any suitable manner in one or more embodiments. In the following description, numerous specific details are provided, such as examples of layouts, distances, network examples, etc., to provide a thorough understanding of embodiments of the invention. One skilled in the relevant art will recognize, however, that the invention can be practiced without one or more of the specific details, or with other methods, components, layouts, etc. In other instances, well-known structures, materials, or operations are not shown or described in detail to avoid obscuring aspects of the invention.

The foregoing detailed description describes the invention with reference to specific exemplary embodiments. However, it will be appreciated that various modifications and changes can be made without departing from the scope of the present invention as set forth in the appended claims. The detailed description and accompanying drawings are to be regarded as merely illustrative, rather than as restrictive, and all such modifications or changes, if any, are intended to fall within the scope of the present invention as described and set forth herein.

1. A mechanically commutated motor configured for use with an external magnetic field, comprising:
an axle comprising a non-magnetic material;
a rotor coupled to the axle, the rotor comprising:
three or more actuator units spaced about the axle, wherein each actuator unit comprises a non-magnetic material;
a coil winding along each of the three or more actuator units; and
a commutator coupled to the axle and electrically associated with one or more of the coil windings; and
two or more resilient contacts oriented to direct a current through the commutator to one of the coil windings to induce a current in the coil winding to form an electromagnet that is configured to rotate the rotor relative the external magnetic field from a magnet located external to the mechanically commutated motor.

2. The mechanically commutated motor of claim 1, further comprising a motor case surrounding the rotor, the motor case comprising a non-magnetic material.

3. The mechanically commutated motor of claim 2, wherein the non-magnetic material of the three or more actuator units and the axle and the motor case have a magnetic susceptibility χ that meets the condition |χ_{w}−χ|<10⁻¹ where χ_{w} is the magnetic susceptibility of water.

4. The mechanically commutated motor of claim 1, wherein the axle is comprised of a low-conducting material that does not radiate electromagnetic noise from the mechanically commutated motor.

5. (canceled)

6. The mechanically commutated motor of claim 1, wherein the magnet located external to the motor is a high field magnet in a magnetic resonance imaging (MM) system.

7. The mechanically commutated motor of claim 1, wherein the magnet located external to the motor is a superconducting magnet.

8. The mechanically commutated motor of claim 1, further comprising power supply terminals coupled to the two or more resilient contacts and configured to be coupled to a power supply to provide a direct current voltage.

9. (canceled)

10. (canceled)

11. The mechanically commutated motor of claim 1, further comprising electromagnetic shielding configured to substantially enclose the motor to reduce electromagnetic interference from the motor to an external device.

12. The mechanically commutated motor of claim 1, wherein each coil winding is comprised of a conductive material that is non-magnetic.

13. (canceled)

14. (canceled)

15. The mechanically commutated motor of claim 1, further comprising a motor controller configured to be coupled to the mechanically commutated motor via a wired connection.

16. The mechanically commutated motor of claim 15, further comprising a high magnetic field compatible position encoder coupled to the axle to provide feedback of a rotation of the rotor to a processor.

17. The mechanically commutated motor of claim 16, wherein the high magnetic field compatible position encoder is configured to communicate a position of the axle to a motor controller to enable the motor controller to generate a driving current in each rotor winding to achieve at least one of a desired position of the rotor in the mechanically commutated motor, and achieve one or more of a predetermined speed or acceleration of the rotor in the mechanically commutated motor.

18. (canceled)

19. The mechanically commutated motor of claim 17, further comprising an unbalanced weight attached to the axle to form a mechanical excitation device configured to be controlled by the motor controller using the high magnetic field compatible position encoder.

20. The mechanically commutated motor of claim 17, wherein the controller is configured to output the driving current used to maintain the position of the axle.

21. The mechanically commutated motor of claim 16, wherein the high magnetic field compatible position encoder comprises an optical sensor configured to send an electronic signal to the motor controller.

22. (canceled)

23. The mechanically commutated motor of claim 20, wherein the high magnetic field compatible position encoder is configured to send a digital signal via a fiber optic cable to the motor controller.

24. The mechanically commutated motor of claim 15, wherein the motor controller comprises an H-bridge circuit.

25. The mechanically commutated motor of claim 15, further comprising one or more radio frequency (RF) traps included in the wired connection to reduce absorption and re-radiation of radio frequency signals by the wired connection.

26. The mechanically commutated motor of claim 25, wherein the one or more RF traps are included in a shield of the wired connection and the one or more RF traps are tuned to attenuate radio frequency signals at the Larmor frequency of a magnetic resonance imaging system.

27. The mechanically commutated motor of claim 15, further comprising a controller shielded enclosure configured to substantially surround the motor controller.

28. The mechanically commutated motor of claim 27, wherein the wired connection is one of a twisted shielded pair and a coaxial cable, and a ground of the wired connection is coupled to a ground of the controller shielded enclosure.

29. The mechanically commutated motor of claim 27, further comprising a motor shielded enclosure configured to substantially surround the motor sufficient to prevent RF noise generated by the controller, the motor, and the wired connections from being radiated outside the mechanically commutated motor, wherein the motor shielded enclosure is electrically coupled to the shield of the wired connection which is electrically coupled to the controller shielded enclosure.

30. (canceled)

31. The mechanically commutated motor of claim 19, wherein the motor controller controls a revolutions per minute of the motor to generate a desired mechanical excitation using feedback from the high magnetic field compatible position encoder.

32. The mechanically commutated motor of claim 31, wherein the desired mechanical excitation is timed with gradient waveforms and imaging protocols to achieve an elastography measurement.

33. The mechanically commutated motor of claim 25, wherein multiple RF traps are spaced apart along the wired connection to reduce unwanted common mode currents.
